(19) United States
(12) Patent Application Publication
(10) Pub. No.: US 2021/0298297 A1
(43) Pub. Date: Sep. 30, 2021

(54) MOSQUITO ATTRACTANTS

(71) Applicants: Wageningen Universiteit, Wageningen (NL); London School of Hygiene and Tropical Medicine, London (GB)

(72) Inventors: Jetske Gudrun de Boer, Wageningen (NL); Ailie Robinson, London (GB); James George Logan, Hertford (GB); Joseph Johannes Antonius van Loon, Bennekom (NL); Willem Takken, Wageningen (NL)

(73) Assignees: Wageningen Universiteit, Wageningen (NL); London School of Hygiene and Tropical Medicine, London (GB)

(21) Appl. No.: 17/041,602

(22) PCT Filed: Mar. 25, 2019
(86) PCT No.: PCT/GB2019/050838

§ 371 Date: Sep. 25, 2020

(30) Foreign Application Priority Data
Mar. 28, 2018 (GB) 1805023.7

(51) Int. Cl. A01N 35/02; A01M 1/02; G01N 33/497

(52) U.S. Cl. CPC A01N 35/02; G01N 2033/4975; G01N 33/497; A01M 1/023

(57) ABSTRACT

A mosquito attractant composition is described comprising heptanal, octanal, nonanal, (E)-2-octenal and (E)-2-decenal. More particularly a composition wherein the compounds are present in the following proportions: nonanal 1.00, octanal 0.32±0.16, heptanal 0.06±0.03, (E)-2-octenal 0.04±0.02 and (E)-2-decenal 0.13±0.065.

### CROSS REFERENCE TO RELATED APPLICATIONS

This is the U.S. National Stage of International Application No. PCT/GB2019/050838, filed Mar. 25, 2019, which was published in English under PCT Article 21(2), which in turn claims the benefit of Great Britain Application No. 1805023.7 filed on Mar. 28, 2018.

### FIELD OF THE INVENTION

The invention relates the fields of chemistry, parasitology, entomology and public health in connection with malaria. In part the invention concerns the detection and diagnosis of malaria infection in individuals, and populations of individuals when data from a multiplicity of individuals is collated. Also in part the invention concerns chemical compostions that can be used as baits or lures to trap mosquitoes which transmit the Plasmodium parasite—which is the cause of malaria—and other diseases including filariasis and arboviruses.

### BACKGROUND

Malaria is a serious tropical disease spread by mosquitoes, which if not diagnosed and treated promptly, can be fatal. Malaria is caused by a eukaryotic blood parasite Plasmodium. There are many subgenera and species of Plasmodium, but only five cause malaria in humans. P. falciparum, P. vivax, P. ovale, and P. malariae together account for nearly all human infections with Plasmodium species, with P. falciparum accounting for the overwhelming majority of malaria deaths. Plasmodium knowlesi is an emerging threat to humans. There are other Plasmodium species that infect primates, non-human mammals, birds, reptiles and lizards.

The Plasmodium parasite is spread by mosquito vectors. Where humans are concerned this is the female Anopheles mosquito, of which there are a number of species. For example, the primary malaria vectors in Africa include An. gambiae, An. funestus and An. arabiensis.

Most Anopheles mosquitoes become active at dusk or dawn (they are crepuscular) or they are nocturnal. Some may feed on human hosts indoors (endophagic), while others feed outdoors (exophagic). Biting by nocturnal, endophagic Anopheles mosquitoes is markedly reduced by using insecticide-treated bed nets, by improved housing construction to prevent mosquito entry, and indoor residual spraying of insecticides. Vectors can also be controlled through destruction of the aquatic breeding sites.

Other methods of mosquito control, often near or in areas of human habitation, include physical traps to which the insects are lured by attractants. In its simplest form, a trap employs an incandescent light as an attractant, with a fan to suck the insects drawn to it into a net. Alternative or additional attractants can be employed in various combinations, such as heat, sound, carbon dioxide and/or chemical lures. There are also different modes of physical capture and/or killing, such as electrocution or sticky traps. A range of traps of various designs and modes of operation are available from commercial suppliers.

Various attractant compounds and compositions have been disclosed for use in traps in order to enhance their effectiveness, efficiency and/or specificity for mosquito species.

WO2004/034783 A2 Universidade Federal De Minas Gerais discloses a mosquito trapping device for avoiding the use of any insecticide and method of capturing ovipositing female mosquitoes of species Aedes aegypti, Aedes albopictus, Anopheles sp. and Culex sp. for example, in order to monitor, detect and control them. The trap is characterized by a dark container with at least one opening and with a total or partially sticky inner surface. In certain trials of the device attractants for mosquitoes are included, such as natural attractants (e.g. infusions of organic material such as grass) or a synthetic oviposition attractant such as decanal and nonanal with p-cresol.

WO2017/060682 A1 London School of Hygiene & Tropical Medicine & Rothamsted Research Ltd discloses a composition and devices containing the composition, but for attracting and controlling bed bugs, not malaria transmitting mosquitoes. The bed bug attractant composition comprises (E)-2-octenal and nonanal. The composition may further comprise hexanal, heptanal, octanal and nonanal.

EP3103332 A Crea discloses heptanal but only in connection with a composition for attracting and trapping cherry fruit fly (Rhagoletis cerasi). Other compositions are disclosed in connection with mosquitoes but for the purpose of repelling not attracting them.

WO2010/102049 A2 The Regents of the University of California discloses compositions for controlling how insects are attracted to subjects. The compositions are not formulated for attracting but rather as insect repellents and/or masking agents by virtue of their property to block a critical component of the host odour cue. The compounds are effective if they are capable of inhibiting the electrophysiological response of the CO₂ neuron in insects, e.g. mosquitoes. The volatile compounds of the disclosure have masking and repellent effects by impairing the ability of the insect to find a host via long-range cues from CO₂ plumes emitted from human breath. The compounds are selected from the group consisting of 4 to 6 carbon aldehydes, e.g., butanal, pentanal, hexanal; 5 to 8 carbon alcohols, e.g., pentanol, hexanol, cyclohexanol, Z-3-hexen-1-ol, Z-2-hexen-1-ol, 1-hexen-3-ol, 1-hepten-3-ol, 3-hexanol, 2-hexanol; and 3 to 8 carbon mono- or di-ketones, e.g. butanedione, (2,3)-butanedione and pentanedione.

CN102125037 A discloses a liquid for trapping Aedes albopictus comprising: 1% (v/v) lactic acid, 1% (v/v) acetone, 1% (v/v) linalool, 1% (v/v) octanal, 1% (v/v) skatole, 1% (v/v) indole, 1% (v/v) nonanal, 1% (v/v) nonylacetate, 1% (v/v) heptylacetate and 1% (v/v) octylacetate. The components are mixed and diluted with distilled water.

PI 0505952-6 A Eiras concerns a mosquito trap having various mixtures of proportions of nonanal and decanal embedded in non-repellent resin for slow and steady release.

US2009/0148399 A1 Bette discloses compositions in a biodegradable carrier for the purpose of attracting female egg-laying mosquitoes. The compositions comprise whether singly or in combination the pheromone heterocyclic diastereoiomeric lactone mixture, (5R,6S)-hexadecanolide, 3-Methyl indole, lactone, epsilon-caprolactone, 6-hexanolactone, 6-pentyl-alpha-pyrone, phenol, p-cresol, 4-ethylphenol, 4-methylphenol, indole, 3-methylindole, nonanal, 2-undecanone, 2-tridecanone, naphthalene, dimethyltrisulfide, dodecanoic acid, tetradecanoic acid, (Z)-9-hexadecanoic acid, hexadecanoic acid, (Z)-9-octadecanoic acid, octadecanoic acid and n-heneicosane. Also the following bacteria/fungus groups and their underlying chemical derivatives; Enterobacter cloacae, Acinitobacter calcoaceticus, Psychrobacter immobilis, Bacillus cereus, Trichoderma viride, Polyporus spp., Aerobacter aerogenes, Sphingobacterium multivorum, Trichodermin, Alamethicin, Trichoviridin or Trichotoxin.

WO2014/113876 A1 Laurentian University concerns compositions for attracting egg laying females of certain mosquito species. The compositions comprise one or more attractants and an N-P-K additive. The attractants may be one or more substances selected from: 1) carboxylic acids and esters, in particular decanoic acid, dodecanoic acid, tetradecanoic acid, tetradecanoic acid methyl ester, hexadecanoic acid, hexadecanoic acid methyl ester or octadecanoic acid, or a combination of one or more, such as, propyl octadecanoate, n-heneicosane, tetradecanoic acid methyl ester; 2) alkyl aldehyde, such as nonaldehyde; 3) amine compound, such as triethylamine; 4) phenol compound, such as p-cresol; 5) indole compounds, such as 3-methylindole and 4-methylindole; and 6) other natural or synthetic mosquito attractants.

WO2010/002259 A1 Wageningen Universiteit discloses an agent derived from bacterial cultures for attracting mosquitoes. The agent comprises one or more compounds selected from the group of 2-hydroxy-3-pentanone and benzene ethanol, and optionally one or more auxiliary volatile organic compounds selected from the group consisting of 1-butanol, 2,3-butanedione, 2-methyl-1-butanol, 2-methylbutanal, 2-methylbutanoic acid, 3-hydroxy-2-butanone, 3-methyl-1-butanol, 3-methylbutanal and 3-methylbutanoic acid. The agent can also comprise an insecticide. Two new compounds produced by the bacterial culture 2,2-hydroxy-3-pentanone and benzene ethanol are disclosed to be attractants, whereas benzaldehyde, furfural and hexanal are disclosed as repellents.

WO20115077843A1 COMMW SCIENT IND RES ORG discloses a method for identifying a subject with a Plasmodium infection. The method comprises detecting one or more volatile organic compounds and wherein the levels of the one or more volatile organic compounds indicate a Plasmodium infection. However, the disclosed method measures volatile organic compounds such as allyl methyl sulphide, I-raethylthiopvopane, (E)-I-methylthio-1-propene and (Z)-I-methylthio-1-propene.

In the life cycle of Plasmodium parasites, these enter the vertebrate host through a mosquito bite. Sporozoites enter the skin and travel through the bloodstream to the liver, where they multiply into merozoites, which return to the bloodstream. Merozoites infect red blood cells, where they develop through several stages to produce either more merozoites, or gametocytes. Gametocytes are taken up by a mosquito and infect the insect, continuing the life cycle. In the life cycle of the Anopheles mosquito, the female always needs a blood meal for the development of eggs.

Changes in attractiveness in both animal and human malaria systems have previously been demonstrated. (See for example Busula, A. O., Bousema, T., Mweresa, C. K., Masiga, D., Logan, J. G., et al. Gametocytaemia increases attractiveness of Plasmodium falciparum-infected Kenyan children to Anopheles gambiae mosquitoes. J. Infect. Dis. 216(3):291-295 (2017)). Changes in vertebrate host attractiveness in response to infection have also been documented in other vector-borne disease systems.

Body odour, comprising the volatile compounds emitted from the skin of vertebrates, is the most important cue used by Anopheles for host location (Takken, W. & Knols, B. G. Odor-mediated behavior of Afrotropical malaria mosquitoes. Annu. Rev. Entomol. 44, 131-57 (1999)). Differences in the composition of body odour have been shown to be responsible for variation in attractiveness to biting insects known to exist between people (see also Logan, J. G., Birkett, M. A., Clark, S. J., Powers, S., Seal, N. J., et al. Identification of human-derived volatile chemicals that interfere with attraction of Aedes aegypti mosquitoes. J Chem Ecol 34, 308-322 (2008) and Verhulst, N. O., Qiu, Y. T., Beijleveld, H., Maliepaard, C., Knights, D., et al. Composition of human skin microbiota affects attractiveness to malaria mosquitoes. PLoS ONE [Electronic Resour. 6, e28991 (2011)). From work described in Fernández-Grandon, G. M., Gezan, S. a., Armour, J. a. L., Pickett, J. a. & Logan, J. G. Heritability of Attractiveness to Mosquitoes. PLoS One 10, e0122716 (2015) and elsewhere, these differences may be influenced by body weight and/or surface area, hormones or genetic factors. Prugnolle, F., Lefèvre, T., Renaud, F., Møller, A. P., Missé, D., et al. Infection and body odours: Evolutionary and medical perspectives. Infection, Genetics and Evolution 9, 1006-1009 (2009) demonstrated how human body odour can also be influenced by disease, including metabolic disorders, genetic disorders, and infections. Another study has found compositional changes in body odour during controlled human malaria infection (CHMI), with a variable effect on attractiveness (see de Boer, J. G., Robinson, A., Powers, S. J., Burgers, S. L. G. E., Caulfield, J. C., et al. Odours of Plasmodium falciparum-infected participants influence mosquito-host interactions. Sci. Rep. 7(1):9283 (2017)).

In animals, a study of Plasmodium infection in mice found such changes in body odour to be associated with changes in attractiveness to mosquitoes (see De Moraes, C. M., Stanczyk, N. M., Betz, H. S., Pulido, H., Sim, D. G., et al. Malaria-induced changes in host odors enhance mosquito attraction. Proc. Natl. Acad. Sci. U.S.A. 111(30), 11079-11084 (2014).

While increased attractiveness of Plasmodium-infected individuals has been demonstrated in a malaria-endemic setting (Busula, A. O., Bousema, T., Mweresa, C. K., Masiga, D., Logan, J. G., et al., Gametocytaemia increases attractiveness of Plasmodium falciparum-infected Kenyan children to Anopheles gambiae mosquitoes. J. Infect. Dis. 216(3):291-295 (2017)), no study has yet investigated the skin chemistry underlying this phenomenon Aldehydes are known to be among the many volatiles that constitute human skin odour (see for example, Penn, D. J., Oberzaucher, E., Grammer, K., Fischer, G., Soini, H. A., et al. Individual and gender fingerprints in human body odour. J. R. Soc. Interface 4, 331-40 (2007)). Ketones are also known volatiles of human skin. Aldehydes are found in the skin odour of various mammalian species and have previously been determined to be among the chemicals used by haematophagous insects for host location (see for example, Puri, S. N., Mendki, M. J., Sukumaran, D., Ganesan, K., Prakash, S., et al. Electroantennogram and Behavioral Responses of Culex quinquefasciatus (Diptera: Culicidae) Females to Chemicals Found in Human Skin Emanations. J. Med. Entomol. 43, 207-213 (2006)).

Becker, K., Tilley, L., Vennerstrom, J. L., Roberts, D., Rogerson, S., et al. Oxidative stress in malaria parasite-infected erythrocytes: Host-parasite interactions. Int. J. Parasitol. 34, 163-189 (2004) have noted how aldehyde and ketone compounds are synthesised when reactive oxygen species attack a lipid-dense membrane structure, i.e. lipid peroxidation, caused by oxidative stress. Oxidative stress is known to characterise malaria infection, occurring in the erythrocytes and liver.

A recent publication found the aldehydes octanal, nonanal and decanal to be among volatile compounds emitted by red blood cell (RBC) cultures that had been supplemented by (E)-4-hydroxy-3-methyl-but-2-enyl pyrophosphate (HMBPP) (see Emami, S. N., Lindberg, B. G., Hua, S., Hill, S., et al. A key malaria metabolite modulates vector blood seeking, feeding, and susceptibility to infection. Science 80 4563 1-9 (2017). HMBPP is a precursor in the 2-C-methyl-D-erythritol 4-phosphate (MEP) pathway, apparently used by Plasmodium for isoprenoid production, and it was suggested that HMBPP triggered enhanced release of these compounds from infected RBC (iRBC), with a subsequent impact on mosquito attraction. Additionally, terpenes were isolated from HMBPP RBC, and another study also isolated terpenes above Plasmodium infected RBC cultures (see Kelly, M., Su, C.-Y., Schaber, C., Crowley, J. R., Hsu, F.-F., et al. Malaria parasites produce volatile mosquito attractants. MBio 6, e00235-15-(2015). Although the MEP pathway is a possible source of terpenes via isoprenoid production in infected RBC, the source of terpenes in HMBPP RBC remains unknown. It should be emphasised that laboratory-based studies of the volatile compounds isolated above iRBC cultures do not characterise the human body odour used by mosquitoes during host location. As such, they do not fully capture the complex biological and biochemical host-parasite interactions that occur in natural Plasmodium infections.

Also important to note that whilst the lipid peroxidation pathway for aldehyde production is well-established, the skin microbiota are also known to produce aldehydes. For example, human feet harbour skin microflora that produce volatiles that are attractive to mosquitoes (Verhulst, N. O., Beijleveld, H., Knols, B. G., Takken, W., Schraa, G., et al. Cultured skin microbiota attracts malaria mosquitoes. Malar. J. 8, 302 (2009)). Differences in microflora have been associated with differences in attractiveness (see Verhulst, N. O., Qiu, Y. T., Beijleveld, H., Maliepaard, C., Knights, D., et al. Composition of human skin microbiota affects attractiveness to malaria mosquitoes. PLoS ONE [Electronic Resour. 6, e28991 (2011).

The inventors studied asymptomatic children in Western Kenya and, using analytical chemistry and the antennal and behavioural responses of Anopheles mosquitoes, identified and established the role of Plasmodium infection-associated compounds (“IACs”) in human body odour. Surprisingly the inventors have discovered how elevated production of certain specific aldehydes in skin odour are associated with increased attractiveness to mosquitoes in Plasmodium-infected humans. The increased production of some of these infection-associated aldehydes was correlated with total parasite density. A generally positive association was discovered between P. falciparum asexual parasite biomass and gametocyte density. The inventors' identification of the particular IACs, with their demonstrated impact on mosquito behaviour, has the practical application in the improvement of functional lures for trapping malaria mosquitoes. Also, to serve as biomarkers for malaria and thereby provide non-invasive diagnostic assays.

### BRIEF SUMMARY OF THE DISCLOSURE

Accordingly, the present invention provides a mosquito attractant composition comprising heptanal, octanal, nonanal, (E)-2-octenal and (E)-2-decenal. Compositions of the invention are advantageously more attractive to mosquitoes than other attractant compositions, because they mimic odours of Plasmodium-infected animals or humans. Mosquitoes are generally more attracted to the odours of Plasmodium-infected individuals than uninfected individuals.

The attractant composition may be provided in various forms, that is to say a concentrated stock solution for storage and transport prior to dilution and use, or for use in manufacture. The attractant composition may be in a ready-to-use formulation and at appropriate concentrations, as defined and explained in more detail later.

The compounds comprised in the composition are:

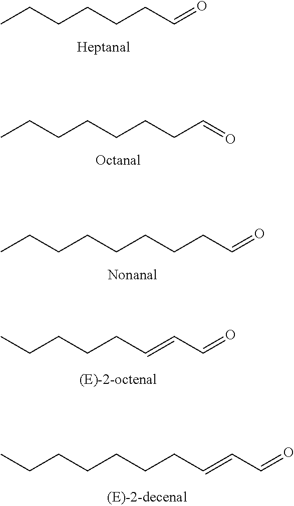

Other compounds or substances may be present in the composition in number and concentration to which they do not detract from the attractant property of the composition as a whole for mosquitoes. For example, these other compounds may serve as carriers, stablilizers or some other function, e.g. insecticide.

Mosquito attractant compositions of the invention preferably have the following ratios of heptanal, octanal, nonanal, (E)-2-octenal and (E)-2-decenal, based on a reference of 1 part nonanal:

| | |
|---|---|
| nonanal | 1.00 |
| octanal | 0.32 ± 0.16 |
| heptanal | 0.06 ± 0.03 |
| (E)-2-octenal | 0.04 ± 0.02 |
| (E)-2-decenal | 0.13 ± 0.065 |

In some compositions hexanal may additionally be present:

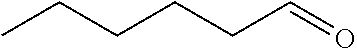

In other compositions, which may include hexanal as well, 1-octen-3-one may be present:

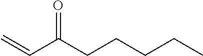

| | |
|---|---|
| Nonanal | 1.00 |
| Octanal | 0.32 |
| Heptanal | 0.07 |
| (E)-2-octenal | 0.05 |
| (E)-2-decenal | 0.15 |
| hexanal | 0.18 |
| 1-octen-3-one | 0.05 |

Mosquito attractant compositions of the invention preferably do not comprise 2-octanone. This compound has been found not to be associated with attractiveness to mosquitoes.

Formulations of the composition of the invention may be aqueous or organic. In the form of a stock solution for further various uses also involving dilution, the composition may comprise a volatile organic solvent as a carrier. These solvents can be alcohols, e.g. ethanol, butanol or other solvents such as hexane or diethyl ether. Dilution of compositions may be using additional similar or other organic solvents and/or water, usually distilled water. The compositions of the invention may be applied to the desired substrate in a solvent which would evaporate off leaving behind the active compounds.

Also, in other embodiments, a substrate may be provided and amounts of each individual component of an attractant blend of the invention may be applied separately, sequentially or simultaneously, to the substrate. The substrate may be adsorbent of the compounds. Upon drying of the compounds to the substrate this emits the required blend of compounds in volatile form.

In another aspect, the invention provides a mosquito attractant composition comprising a natural human or animal odour source plus added heptanal. This is an alternative mode of operating the invention whereby a natural human or animal odour, usually collected directly from a human or animal who is not Plasmodium-infected, or who has a low level of infection or non-gametocyte stage. The odour is augmented with the heptanal in order to mimic the odour as being from an infected individual or an individual or greater infection.

Also provided by the invention in a further aspect is a natural or synthetic mosquito attractant odour composition, whether in liquid or volatile form, further comprising heptanal. The heptanal may be present at a concentration of about 1×10⁻⁸ g/ml+0.5×10⁻⁸ g/ml; optionally +0.25×10⁻⁸ g/ml or +0.10×10⁻⁸ g/ml. When the composition is in volatile form, the heptanal may be present in an amount of at least 0.7% (v/v) optionally at least 0.9% (v/v) of all volatile odour compounds present. Again, the heptanal is used to augment the odour or liquid or other composition so that it mimics a Plasmodium-infected human or animal. The heptanal may be present in an amount selected from (v/v) of all volatile compounds: at least 0.8%, at least 0.9%, at least 1.0%, at least 1.1%, at least 1.2%.

Alternatively, or additionally (in combination with any of the above lower limits of heptanal relative to volatile odour compounds, the upper limit (v/v) for heptanal may be selected from: not more than 1.5%, not more than 1.4%, not more than 1.3%, not more than 1.2% or not more than 1.1%.

Any of the compositions of the invention hereinbefore defined may be provided in gaseous form, e.g. in a canister with propellant to form a spray. Naturally, the propellant should have minimal influence on mosquito behaviour and reactions to the odour compositions of the invention.

In another aspect, the invention provides a mosquito trapping composition. Such compositions are useful in connection with luring or baiting mosquitoes, whether to immobilise, trap and/or kill. A certain trapping composition comprises a non-drying sticky or adhesive substance and any of the attractant compositions as hereinbefore defined. Such compositions may be used as treatments which can be painted or sprayed onto surfaces, e.g. walls of houses or rooms, so as to capture mosquitoes to those surfaces. These compositions may be intermediates in a manufacturing process for making solid phase surfaces for insertion into existing mosquito traps. In specific example a fly paper for attracting and trapping mosquitoes may be made.

Any of the compositions of the invention as hereinbefore defined may include an insecticide for killing mosquitoes. Suitable examples of such insecticides include malathion, resmethrin, sumithrin or permethrin.

Also included within the invention are apparatuses or devices for trapping and/or killing mosquitoes comprising an attractant composition as hereinbefore defined. The devices may be “lure and kill” for example electrocution devices, or “trap and kill” e.g. an insecticide laced paper in a CO₂ emitting device also including a composition of the invention.

Therefore the invention naturally includes corresponding methods of luring and trapping mosquitoes using the attractants of the invention in any suitable known trapping device. Also, the invention includes corresponding methods of luring and killing mosquitoes using the attractants of the invention together with or as part of any suitable known mosquito killing apparatus.

Advantageously, the attractant compositions of the invention are useful for luring many different sorts of mosquitos, and lures may be used in connection with traps whether passive or for killing mosquitos. Within the term “mosquito” is an insect which is a member of the family Culicidae. This includes the subfamilies Anophelinae and Culicinae; and thereby species of the genera Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, lsostomyia, Johnbe/kinia, Kimia, Limatus, Lutzia, Malaya, Mansonia, Maorigoe/dia, Mimomyia, Onirion, Opifex, Orthopodomyia, Psorophora, Runchomyia, Sabethes, Shannoniana, Topomyia, Toxorhynchites or Trichoprosopon.

In preferred aspects the invention is useful for luring species of mosquito such as Anopheles gambiae complex: Anopheles gambiae s.s, Anopheles gambiae colluzzii (M), Anopheles gambiae gambiae (S); and M/S hybrids. Other species for which the invention is useful is Culex sp; e.g. C. quinquefasciatus and C. tritaenoiorhynchus. Also, Aedes aegypti; e.g. Aedes aegypti aegypti, or Aedes formosus.

The invention also provides a method of detecting Plasmodium infection in a subject comprising: collecting a sample of odour emanated from the subject, detecting and measuring amounts of one or more indicative volatile compounds in the odour, the indicative volatile compound(s) selected from: heptanal, octanal, nonanal, (E)-2-octenal and (E)-2-decenal, 2-octanone, hexanal or 1-octen-3-one; comparing the measured amounts of the indicative volatile compounds with: i) the amounts of the same compounds in a reference sample of body odour from an uninfected subject or subjects; and/or ii) predetermined reference amounts equating to uninfected individual status; wherein an increase in the indicative volatile compound(s) indicates the subject has a Plasmodium infection.

The reference sample of odour can be from a single Plasmodium-uninfected person or can be an aggregate of odours from a plurality of such uninfected persons. The reference odour amounts may be values measured using gas chromatography (GC) analysis in similar way to that described in the following examples. Reference odour amounts can be an amount in ng for a given standardised volume, e.g. 100 ml of sample air, or an amount in ppm, for example. The reference sample amounts can be run sequentially with the enquiry samples on the same machine. Alternatively the odour component values can be simply stored in computer readable medium and accessed by a computer used to process the GC measurement data. A computer program can be used to provide sample odour component values comparison with reference values and from this provide indication as to whether or not predefined threshold values are found in the sample. Attaining or exceeding the defined threshold value amounts by the sample odour components compared to reference being determinative of infection status of the individual who has provided the test sample.

Also provided by the invention is a method of detecting Plasmodium infection in subject animals or humans, preferably in humans, wherein the indicative volatile compounds are (E)-2-octenal and (E)-2-decenal and an increase in the amounts or values of (E)-2-octenal and/or (E)-2-decenal measured in a test sample and compared to reference sample and/or reference amounts indicates that the subject is Plasmodium positive.

In some embodiments of the aforementioned method, the amount of increase in heptanal, octanal and nonanal compared to reference sample and/or reference amounts is proportional to the Plasmodium infection density in the subject. Such infection density may be expressed as number of parasites per μl of blood.

In another optional embodiment of the aforementioned method of the invention, the indicative volatile compound may be selected from one or more of 2-octanone, hexanal and 1-octen-3-one and an increase in the indicative compound(s) compared to reference sample(s) thereof, and/or reference amount(s) is indicative of the presence of microscopic gametocytes in the subject. These may be expressed as number of gametocytes per μl of blood.

In practice, diagnostic aspects of the invention may be put into effect by using apparatus such as mass spectrometer (MS), gas chromatography (GC), GC-MS, e-nose, z-nose, a molecular chip. Also possible is to train a dog to react to the odour(s) which are described herein as being emitted by a Plasmodium infected individual. In this way a suitably trained dog can be employed as a “sniffer” for likely Plasmodium infected individuals who could then be further tested for infection status.

### BRIEF DESCRIPTION OF THE DRAWINGS

Embodiments of the invention are further described hereinafter with reference to the accompanying drawings, in which:

FIG. 1 shows the effect of parasitological status on Anopheles gambiae sensu strictu (s.s.) preference for body odour sampled at two time points, one during Plasmodium infection (T1) and the other following parasite clearance (T2). Light bars represent attraction to odour from parasite-free samples, dark bars represent attraction to odour samples from individuals with parasites. Groups of ten mosquitoes were given a choice between socks worn by each participant at both T1 and T2, in a dual choice cage assay, with the number of mosquitoes that chose the T1 or T2 odour sample being summed over six replicates per participant. Participants were grouped into those with gametocytes by microscopy or QT-NASBA (at >50 gametocytes/μL) (n=23), those with asexual stages only by microscopy (n=10), or parasite-free (n=12). Of those with asexual parasites, three had sub-microscopic gametocytes (1-34.9 gametocytes/μL blood), and three were not tested. Predicted mean proportions from the GLM are plotted with 95% CI, and significant differences from 0.5 are indicated with * (P<0.05) (GLM included infection status only as predictor of proportion of mosquitoes attracted to T1 odour samples).

FIG. 2 shows a schematic drawing of the cage assay used to test the effect of parasitological status on Anopheles gambiae sensu stricto (s.s.) preference for body odour sampled at two time points, one during Plasmodium infection (T1) and the other following parasite clearance (T2). Two mosquito cages wrapped with kitchen cling-film were connected using three WHO bioassay tubes, with slide units between the inner and outer tubes. Each cage contained a pair of socks, with samples from the same child, collected during infection or after antimalarial treatment, offered in a dual-choice situation. Ten female mosquitoes were released in the central tube and given 15 minutes to fly to either cage.

FIG. 3 shows a schematic diagram of protocol (top half of image) for odour sampling by air entrainment from Plasmodium-infected individuals, for use in GC-EAG analysis (bottom half of image, here with Anopheles coluzzii) and direct GC analysis of entire odour profile. Children were recruited for odour sampling in groups of three to represent parasite-free, asexual parasite carriers, and gametocyte carriers, if parasite prevalence allowed. Following malaria diagnosis by point-of-care methods and odour sampling, malarious individuals were treated, and the same cohort re-sampled on days 8 and 22. Whole blood samples were also taken for retrospective molecular analysis. During GC-EAG, odour samples are injected by syringe at the inlet directly into the column (1), where they are vaporised, and carried through the column by the carrier gas (here hydrogen) (2). During passage through the (50 m) HP1 column, constituents of the sample are separated by gas chromatography, and analytes are split as they elute from the column (3). A proportion is directed, via a heated transfer line (4), into a humidified, purified, airflow (5), which is then directed over the insect antennae (6), simultaneously to the proportion that is detected by a flame ionisation detector on the GC (7). GC analytes are represented by peaks (top; GC trace) while antennal response by nerve cell depolarisation causes a perturbation in the electroantennographic detection (EAD trace), indicating entomologically significant analytes.

FIGS. 4A-L: (A)/(B)/(C) heptanal; (D)/(E)/(F) octanal; (G)/(H)/(I) nonanal; (I) (E)-2-octenal; (J) (E)-2-decenal; (K) 2-octanone production (relative to all compounds in odour sample) per group (100-minute odour profile sampling). Predicted means (+SE) given by linear mixed modelling (REML). Sample size in bar ends, *, † significant pairwise difference in mean amount between two groups indicated, tested by Least Significant Difference (P<0.05). A, D, G, J, K, and L, ‘total density’ categorisation: ‘Neg’=negative, ‘lower’ and ‘higher’ refer to parasite densities of lesser or greater than 50 p/μL, ‘Gam’=microscopic gametocytes (B, E, H) ‘quartile’ categorisation; ‘Neg’ and ‘Gam’ as before, L=low, mean/median parasite density 0.38/0.3, n=21; M-L=medium-low, mean/median parasite density 16.77/8.3, n=17; M-H=medium-high, mean/median parasite density 296.60/214.18, n=19; H=high, mean/median parasite density 102669.46/13304.54, n=23. For bar charts CNL(A)=solvent control, CNL(B)=empty bag control. (C)/(F)/(I) show raw gas chromatography output for heptanal, octanal and nonanal. Individual traces represent odour samples, coloured according to the parasitological status of the individual from whom the odour sample was taken, ‘Higher density’, ‘lower density’, and ‘negative’ definitions as above. Gametocyte carriers are excluded for clarity, as compound production spanned higher and lower parasite density groups.

FIGS. 5A-B: (A) Representative GC traces from an individual with a ‘high density’ infection (>50 p/μL blood) and ‘low density’ infection (<50 p/μL blood). Compounds found to be associated with infection (other than 2-octanone, not visible due to very small amounts) are annotated. (B) The proportion (%) that IAC contributed towards the entire odour profile, grouped by parasitological category (‘total density’ categories). The average number of non-IAC per group (i.e. ‘all other volatile compounds’, grey bar), was 171.27 (SE=5.23) across all groups.

FIG. 6 shows Anopheles coluzzii responses in a dual-port olfactometer to heptanal and a blend of five infection-associated aldehydes, ‘Plas 5’. Heptanal (10 μL) at two concentrations (g/mL) was presented with (diagnonal hatch bars), and tested against (diamond hatch bars), odour (socks) from parasite-free study participants (5-12 year-old Kenyan children) over eight replicates. Plas 5 (heptanal, octanal, nonanal, (E)-2-octenal and (E)-2-decenal) at four concentrations (10 μL of 100% approximating the amounts found in the foot odour samples) was presented with (darker bars), and tested against (lighter bars bars), the synthetic lure MB5 (ammonia, (S)-lactic acid, tetradecanoic acid, 3-methyl-1-butanol and butan-1-amine) over 10/11 replicates. Each replicate tested 30 mosquitoes. Predicted mean proportions and 95% CI are presented, from two separate GLMs (for heptanal and Plas 5 assays), assuming a Binomial distribution and using a logit link function. Significant differences from 0.5 are indicated with * (P<0.05) (See Table E7 for details of the GLMs).

FIGS. 7A-D: (A) ‘Total density’ parasitological categorisation, showing actual Plasmodium parasite densities p/μL) per group (Lower, total <50 p/μL blood; Higher, total >50 p/μL blood; Gam, gametocyte carriers by microscopy). Here total parasites (all stages) are shown. Note, of Gametocyte category samples, 65% harboured total parasite densities of >50 p/μL (higher category). Colours represent the diagnostic technique used to inform categorisation. (B) ‘Quartile’ parasitological categorisation, showing actual parasite densities (p/μL) per group. Here all ‘higher density’ and ‘lower density’ samples from the ‘total density’ categories were re-classified: L=low, mean/median parasite density 0.38/0.3, n=21; M-L=medium-low, mean/median parasite density 16.77/8.3, n=17; M-H=medium-high, mean/median parasite density 296.60/214.18, n=19; H=high, mean/median parasite density 102669.46/13304.54, n=23. Colours represent the diagnostic technique used to inform categorisation. (C) Boxplots of median gametocyte densities per μL blood (on log-scale, boxes representing the interquartile range, median being the bottom of the box for ‘Lower’ and ‘Higher’ or the line in the box for ‘Gametocytes’), in ‘total density’ categories (measured by QT-NASBA or microscopy) (D) Correlation in parasite density as measured by 18S qPCR vs. PgMET qPCR, the latter amplifying from either a Whatman filter paper dried blood spot template (wDBS), or a used rapid diagnostic test template (uRDT) for when wDBS was unavailable. Correlations shown only for individuals in the ‘Lower’ and ‘Higher’ density group (A).

FIG. 8: Anopheles coluzzii responses in a dual-port olfactometer to infection-associated compounds (IAC) alone and in a blend, ‘Plas 6’ (dark bars), relative to background odour alone (light bars). IAC at two concentrations (g/mL) were presented with, and tested against, odour (socks) from parasite-free study participants (5-12 year-old Kenyan children), over 8/9 replicates. Plas 6 (heptanal, octanal, nonanal, (E)-2-octenal, (E)-2-decenal and 2-octanone) was presented with, and tested against, the synthetic lure MB5 (ammonia, L-(+)-lactic acid, tetradecanoic acid, 3-methyl-1-butanol and butan-1-amine), at four concentrations and over 10/11 replicates. Each replicate tested 30 mosquitoes. Predicted mean proportions and 95% confidence intervals (CI) are presented, from five generalized linear models (GLMs) assuming a Binomial distribution and using a logit link function. No treatments were preferable to controls, with all 95% CI including 0.5.

FIG. 9 is data of Example 3 and shows mean abundances of mosquitoes. The highest mean trap catch for Anopheles females was MB5+1% Plas 5. Treatment 1: Blank control; treatment 2: MB5 blend; treatment 3: MB5+Plas5 blend 0.1%; treatment 4: MB5+Plas5 blend 1%; treatment 5: MB5+Plas5 blend 10%.

FIG. 10 is data of Example 3 showing the proportion of genus-gender groups in the total trap catches for each of the lure blends. Treatment 1: Blank control; treatment 2: MB5 blend; treatment 3: MB5+Plas5 blend 0.1%; treatment 4: MB5+Plas5 blend 1%; treatment 5: MB5+Plas5 blend 10%.

FIG. 11 is data of Example 3 and shows the species of the Anopheles gambiae s.l complex caught in the trapping period. Treatment 1: Blank control; treatment 2: MB5 blend; treatment 3: MB5+Plas5 blend 0.1%; treatment 4: MB5+Plas5 blend 1%; treatment 5: MB5+Plas5 blend 10%.

### DETAILED DESCRIPTION OF THE INVENTION

The invention will now be described in more particular detail and with reference to Examples.

Alternative mosquito attractant compositions of the invention may have the following ratios of heptanal, octanal, nonanal, (E)-2-octenal and (E)-2-decenal, based on a reference of 1 part nonanal as follows:

| | |
|---|---|
| nonanal | 1.00 |
| octanal | 0.32 ± 0.08 |
| heptanal | 0.06 ± 0.015 |
| (E)-2-octenal | 0.04 ± 0.01 |
| (E)-2-decenal | 0.13 ± 0.0235 |

| | |
|---|---|
| nonanal | 1.00 |
| octanal | 0.32 ± 0.04 |
| heptanal | 0.06 ± 0.0075 |
| (E)-2-octenal | 0.04 ± 0.005 |
| (E)-2-decenal | 0.13 ± 0.0118 |

| | |
|---|---|
| nonanal | 1.00 |
| octanal | 0.32 |
| heptanal | 0.06 |
| (E)-2-octenal | 0.04 |
| (E)-2-decenal | 0.13 |

Attractant blends in accordance with the invention are defined in the table below as follows, whereby the variation (±) is the acceptable range of concentration Wimp of each component compound.

| | | | Optional | Optional | Optional | Optional | Optional | Optional | Optional | Optional |
|---|---|---|---|---|---|---|---|---|---|---|
| | Concentration | Variation | variation | variation | variation | variation | variation | variation | variation | variation |
| | (μg/ml) | (±) | I (±) | II (±) | III (±) | IV (±) | V (±) | VI (±) | VII (±) | VIII (±) |
| Nonanal | 47.6 | 11.9 | 9.52 | 7.14 | 4.76 | 2.38 | 0.1904 | 0.1428 | 0.0952 | 0.0476 |
| Octanal | 15.429 | 3.8575 | 3.086 | 2.3145 | 1.5429 | 0.7715 | 0.61716 | 0.46287 | 0.30858 | 0.15429 |
| Heptanal | 2.857 | 0.71425 | 0.5714 | 0.42855 | 0.2857 | 0.14285 | 0.11428 | 0.08571 | 0.05714 | 0.02857 |
| 2-octenal | 1.871 | 0.46775 | 0.3742 | 0.28065 | 0.1871 | 0.09355 | 0.07484 | 0.05613 | 0.03742 | 0.01871 |
| 2-decenal | 6.136 | 1.534 | 1.2272 | 0.9204 | 0.6136 | 0.3068 | 0.24544 | 0.18408 | 0.12272 | 0.06136 |

Regarding hexanal, the amount or concentration of this may be about 0.16 to 0.17 of the respective amount or concentration of nonanal in a composition.

A mosquito attractant composition as described herein may have heptanal, octanal, nonanal, (E)-2-octenal and (E)-2-decenal present in combination as not more than 60% v/v of total volatiles in the composition. Optionally, not more than a percentage value selected from 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58% or 59%.

However, what is disclosed herein is the possibility that for one of the compounds a certain optional variation in concentration may be chosen, whereas for another compound, a different optional variation concentration may be chosen. That is to say, whilst all compounds may vary in concentration according to the same variation column, this is not necessarily essential. A person of average skill can choose a concentration variation column for each compound in the blend independently of the other, so for example the following is possible where the variations denoted with an asterisk (*) for each compound are chosen:

| | | | Optional | Optional | Optional | Optional | Optional | Optional | Optional | Optional |
|---|---|---|---|---|---|---|---|---|---|---|
| | Concentration | Variation | variation | variation | variation | variation | variation | variation | variation | variation |
| | (μg/ml) | (±) | I (±) | II (±) | III (±) | IV (±) | V (±) | VI (±) | VII (±) | VIII (±) |
| Nonanal | 47.6 | 11.9 | 9.52 | 7.14 | 4.76 | 2.38 | 0.1904 | 0.1428 | 0.0952 | 0.0476 |
| Octanal | 15.429 | 3.8575 | 3.086 | 2.3145 | 1.5429 | 0.7715 | 0.61716 | 0.46287 | 0.30858 | 0.15429 |
| Heptanal | 2.857 | 0.71425 | 0.5714 | 0.42855 | 0.2857 | 0.14285 | 0.11428 | 0.08571 | 0.05714 | 0.02857 |
| 2-octenal | 1.871 | 0.46775 | 0.3742 | 0.28065 | 0.1871 | 0.09355 | 0.07484 | 0.05613 | 0.03742 | 0.01871 |
| 2-decenal | 6.136 | 1.534 | 1.2272 | 0.9204 | 0.6136 | 0.3068 | 0.24544 | 0.18408 | 0.12272 | 0.06136 |

Any other variations denoted with an asterisk (*) like the above to exemplify compositions of the invention are possible.

A mosquito attractant composition of the invention may be part of, or added to a synthetic human or animal mosquito attractant blend. An example of a suitable synthetic attractant blend is BG-Lure mixture of ammonia, lactic acid and caproic acid (Biogents AG, Regensburg, Germany). Also Mbita Blend 5 (MB5) which is described in more detail in Menger D. J., Otieno B., de Rijk M., Mukabana W. R., van Loon J. J., Takken W. (2014) A push-pull system to reduce house entry of malaria mosquitoes. Malar J. 13:119. MB5 includes ammonia (2.5%), lactic acid (85%), tetradecanoic acid (0.00025%), 3-methyl-1-butanol (0.000001%) and 1-butylamine (0.001%).

In the aspects of the invention which provide a mosquito attractant composition comprising a natural human or animal odour source plus added heptanal, the added heptanal may be present providing a total amount of heptanal which is at least about 7% more, 8% more, 9% more, 10% more, 11% more, 12% more, 13% more, 14% more, 15% more, 16% more, 17% more, 18% more, 19% more, 20% more, 21% more, 22% more, 23% more, 24% more, 25% more, 26% more, 27% more, 28% more, 29% more or 30% more than the amount of heptanal in a natural human or animal odour obtained from Plasmodium-free human(s) or animal(s).

A “mosquito attractant” as described herein, is a substance or more particularly a blend of substance molecules which when interact with the sensory apparatus of mosquitoes causing them to move towards a site or area, and usually the source of the substance molecules in air would take the form of a vapour concentration gradient.

The formulations of the invention can be placed in any suitable container or device for dispensing the attractant compound and attracting or trapping mosquitoes.

The attractant compound(s) of this invention may be employed in any formulation suitable for dispensing attractant effective amounts of the compounds. The compounds will generally be employed in formulations comprising a suitable vehicle or carrier containing the attractant compounds.

An attractant composition of the present invention may be applied with a carrier component or carrier (e.g., biologically or agronomically acceptable carrier). The carrier component can be a liquid or a solid material. As is known in the art, the vehicle or carrier to be used refers to a substrate such as a membrane, hollow fiber, microcapsule, cigarette filter, gel, polymers, septa, or the like. All of these substrates have been used to release insect attractants in general and are well known in the art. Suitable carriers are well-known in the art and are selected in accordance with the ultimate application of interest. Solid carriers such as clays, cellulose-based and rubber materials and synthetic polymers may be used.

For example, an attractant composition can be formulated into a waxy medium or vehicle engineered to release desired amounts of vaporous attractant compound at ambient temperatures. Exemplary waxy media are available from Koster Keunen of Watertown, Conn., U.S.A., e.g. Insect Repellent Wax Bar No. 9. This is made of fatty acids ranging in carbon chain length of from C₁₆ to C₂₂, fatty alcohols ranging in carbon chain length of from C₁₆ to C₂₂, paraffinic hydrocarbons ranging in carbon chain length of from C₁₉ to C₄₇, branched hydrocarbons ranging in carbon chain length of from C₂₃ to C₆₉, beeswax and other natural waxes such as candelilla and carnauba. The wax formulations together with the compositions of the invention can be made to have a congealing point in the range from about 75° C. to about 45° C.

Other suitable carriers may include existing mosquito or other insect attractant compositions. The attractant composition of the invention may therefore be mixed with the other attractant composition or compositions. For example, the attractant composition of the invention may be mixed with the art known MB5 attractant composition. The percentage by volume of the attractant composition of the invention to the other attractant composition, e.g. MB5, may be in the range 0.5% (v/v)-99% (v/v). Optionally in the range 1% (v/v) to 50% (v/v). For example, percentage by volume of the attractant composition of the invention to the other attractant composition may be (in v/v) 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%; or any range of percentage between any selected lower % and higher % number in the aforementioned list.

The compositions of the invention may be placed in any suitable container or device for dispensing the attractant compound and attracting or trapping mosquitoes. A “lure and kill” format of device for example. The formulations can be placed in a suitable device so that one can obtain, for example, evaporation of the attractant compound from a porous medium or wax-like medium containing the attractant compound positioned within the dispensing device. As examples of such devices, there can be mentioned the devices disclosed in U.S. Pat. Nos. 5,205,064, 5,799,436 and 6,055,706 of BioSensory Insect Control Corporation and James Nolen & Company. The formulations can also be placed in jar traps such as those that dispense carbon dioxide as an attractant. The formulations can also be placed in “bug zapping” devices for electrocuting the mosquitoes attracted to the device containing the attractant-containing formulation.

Other suitable means of dispensing the attractant compositions of the invention may be by atomization and/or ionic dispersion of the compound as suitable-sized, positively-charged droplets from a suitable atomization or ionic dispersing apparatus, such as the Ionic Wind™ device, available from Brandenburg, Ltd. of Brierery Hill, United Kingdom used in connection with any suitable mosquito trapping device or apparatus.

### Example 1

Materials and Methods

Ethics

Study participants were five- to twelve-year-old children local to the Thomas Odhiambo Campus of icipe in Western Kenya (000251S, 34° 131E), including Rusinga Island, in Suba District, Homabay County. Participants were recruited after obtaining signed consent. The study protocol (NON SSC 389) was approved by the Scientific and Ethical Review Committee of the Kenya Medical Research Institute (KEMRI/RES/7/3/1). Subsequent analyses were conducted at the London School of Hygiene & Tropical Medicine (LSHTM) (ethics reference 8510).

Attractiveness of ‘Infected Odour’ (Socks) by Cage Assays

A cohort of Plasmodium-infected, asymptomatic (tympanic temperature <37.5° C.), individuals that participated in an olfactometer study⁷ was studied for the attractiveness of their skin odour to Anopheles gambiae s.s. Forty-five children were included, of which there were: 23 with microscopic gametocytes or an estimated gametocyte density above 50 gametocytes/μL blood by QT-NASBA, 10 positive for asexual parasites by microscopy, and 12 that tested Plasmodium-parasite free by 18S-qPCR²³. Samples were collected at two time points: within 24 hours of antimalarial treatment but while children still harboured parasites²² (time point one [T1] samples), and 21 days later (time point two [T2] samples). Antimalarial treatment with artemether-lumefantrine (AL) was administered to Plasmodium positive individuals according to manufacturer's instructions (20 mg artemether/120 mg lumefantrine per tablet, Coartem™; Novartis, Basel), and socks were put on within one hour of treatment. Age, haemoglobin (Hb), weight and temperature were measured as covariates⁷. At day 21 (‘after’), both parasitological testing and participant covariate measurements were repeated.

Procedures for Collection of Body Odour

Body odours were collected for 20 hours on nylon socks (97% polyamide, 3% elastane, 20 denier, Hema, The Netherlands), which were washed using 70% ethanol and dried at 70° C. for two hours before use. Surgical gloves were worn throughout collection procedures. Children were assisted in putting on and removing the socks. These were stored in clean glass jars at −20° C. until use in cage assay experiments. Children were asked not to bathe during this time but had no other behavioural restrictions.

Behavioural Assays for Attractiveness of Odour Samples

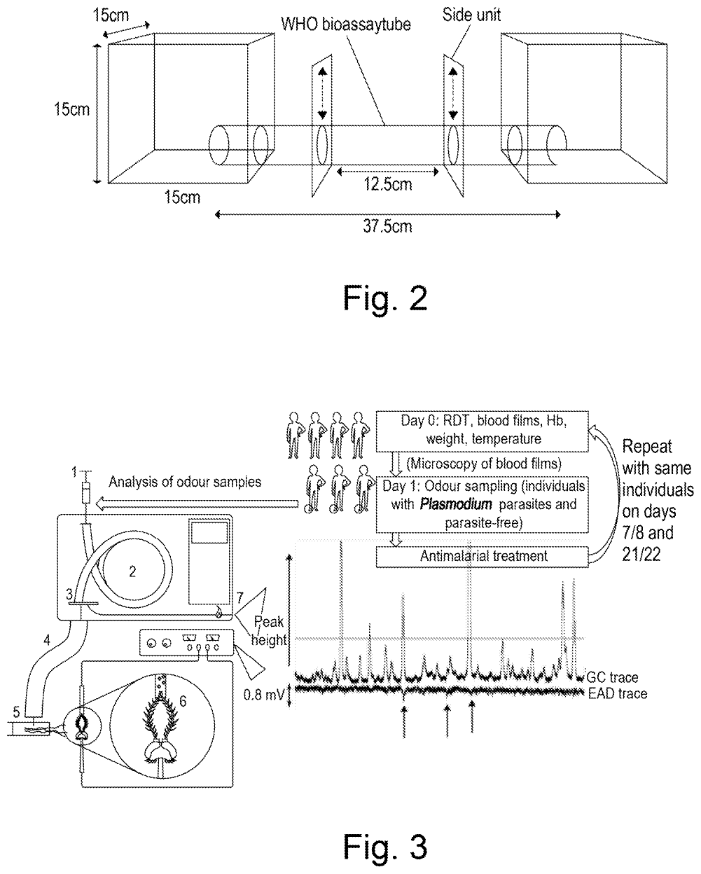

A dual choice cage assay was modified⁴⁷ to determine the relative attractiveness of odour samples from T1 and T2. Three WHO bioassay tubes (12.5 cm long, 5 cm wide)⁴⁸ were connected with sliding units between the inner and outer tubes (FIG. 2). Mosquito cages (15×15×15 cm) were wrapped with transparent kitchen cling-film (Chandaria industries Ltd., Kenya), to prevent movement of volatiles between different assays running in parallel. The outer tubes were inserted six cm into the cages. Per individual, T1 and T2 samples (sock pairs) were placed in opposing cages, with the feet cut off to remove environmental soiling.

Six- to eight- day old, non blood-fed, female An. gambiae s.s. mosquitoes (Mbita strain, with published rearing methods⁴⁹) were collected prior to the experiment and allowed eight hours acclimatisation. Ten mosquitoes were released into the central tube per bioassay, and the gates of the tubes opened for 15 minutes to allow mosquitoes to make a choice of odour source. Experiments were conducted between 18:30-22:30 under ambient conditions, in a red fluorescent-lit room (average temperature, 24.1° C.) with the dual cage covered by black cotton cloth. After 15 minutes mosquito choice was recorded. All sock pairs were tested simultaneously on the same nights, and in total each pair (child) was tested six times, replicating over experimental nights, dual cage set-ups and between cages. All disposable equipment was changed, and cages cleaned (70% ethanol), between experiments/replicates.

Statistical Analysis

Per child, the number of mosquitoes that chose the T1 or T2 odour sample was summed over six replicates, and the relative attractiveness of samples determined as the proportion of mosquitoes that selected a sample over the total number of mosquitoes that made a choice. A generalized linear model (GLM; Binomial distribution, logit link function and dispersion estimated) was used to test the effect of parasitological status (parasite free, asexual or gametocytes) on the relative attractiveness. The number of mosquitoes caught in the cage with the T1 sample was used as the response variable, and all mosquitoes caught in both cages as the binomial total. Covariates associated with participants (age, sex, Hb and tympanic temperature measured at T1) were tested, but removed from the model because they were not significant (P>0.05, F-tests). Per parasitological group, we used the 95 CI of the predicted proportion of mosquitoes choosing T1 odour samples, derived from the GLM, to assess whether mosquito choice differed significantly from a 50:50 distribution over the two odour samples. SPSS® (2016, version 24, IBM) was used for the analyses.

Collection of Volatile Odour Samples

In the same locality, a separate cohort of schoolchildren, of varying Plasmodium infection status, were sampled for foot odour (FIG. 3, top half). On day zero, twenty children for whom the parent or guardian had given full consent were tested for their malaria status by rapid diagnostic test (RDT) and microscopy. Tympanic temperature, age, weight and Hb levels were recorded. Symptomatic children and/or those with a temperature >37.5° C. with RDT positivity were treated with AL (as above), and excluded from the study. Overnight, microscopy was conducted and three children were selected for odour sampling, with the intention to sample one child with asexual parasites, one with gametocyte stages, and one with no parasites. On day one, odour sampling was conducted by air entrainment, after which all malarious children were treated. Days zero and one constituted round one (R1), and the same procedures were conducted at days seven and eight (R2), and 21 and 22 (R3), with the intention to repeat sample the same children at two points post-treatment (FIG. 3). R1-R3 were repeated for six months between January and June 2014. In this way, 56 children were repeat sampled, but a total of 117 odour samples, and 59 accompanying empty bag control samples, was achieved, due to loss-to-follow-up.

For each child, one foot was placed in a prepared bag (Fresh and Eazy oven bags, 45×50 cm, Meda-Pak, Uithoorn, The Netherlands), clipped shut around the calf. At each sampling round (R1-R3), a control (empty) bag was tightly closed and sampled in the same manner. Bags were fitted with Swagelok fittings at opposing corners, allowing connection to polytetrafluoroethylene (PTFE) tubing for air flow. Air (charcoal-filtered) was pumped into the top of the bag and vacuumed from the bottom (both at 500 mL/min), with a 30-minute purge prior to fitting the polymer filters, to ensure system cleanliness. Porapak filters were connected (Porapak Q, mesh size 50/80, Supelco Analytical, Bellefonte, Pa., USA) and sampled for 100 minutes, then stored in stoppered glass vials in a cool box before sealing under filtered nitrogen on the same day. Ampoules were stored at −20° C. until shipping to LSHTM. Prior to use, all PTFE tubing, Swagelok fittings and glassware were cleaned with 70% ethanol, then baked in an oven at 150° C. for two hours. Sampling bags and charcoal filters were baked in the same manner. Cotton gloves were worn by the investigators throughout.

Infection Status

Odour sampling was informed by RDT (One Step malaria HRPII and pLDH antigen rapid test [SD BIOLINE, Cat no 05FK60]), performed as per manufacturer's guidelines, and thick and thin blood films made using peripheral blood from a finger prick. Whole blood (50 μL) was stored in RNAprotect (250 μL; QIAGEN, Germany). Retrospectively, DNA/RNA extraction was performed using Total Nucleic Acid Isolation Kit (with methods as published previously⁵⁰) and P. falciparum parasite density, and stage V gametocyte density, determined by 18S qPCR²³ and QT-NASBA⁵¹. Additionally, dried blood stored on both Whatman No. 3 filter paper (Whatman, Maidstone, United Kingdom [wDBS]) and used RDTs (air dried and stored in sealed plastic bags containing the desiccant silica gel [uRDT]) was used as a DNA template. DNA was extracted from circles (3 mm) punched from the wDBS, and sections (3×2 mm) cut from the central section of the nitrocellulose strips in the uRDTs⁵². Extraction was performed in a deep well plate using an automated extraction system (QIAsymphony), with the QIAsymphony DSP DNA mini kit (QIAGEN, Germany) and according to the manufacturer's instructions, and a Plasmodium tRNA methionine-based duplex qPCR was used to measure Plasmodium density²². Good correlation in parasite density was obtained between duplex qPCR using wDBS or uRDT whole blood template⁵³. Where available, the same DNA extracts were used for species specific (P. falciparum, P. ovale spp. and P. malariae) nPCR⁵⁴, with some P. ovale spp. identifications confirmed by the P. ovale spp. tryptophan-rich antigen (PoTRA) assay⁵⁵.

Gas Chromatography-Electroantennography (GC-EAG) of Pooled Odour Samples

GC-EAG Odour Sample Blends

Porapak filters were eluted using re-distilled diethyl ether (750 μL), and, to approximate an “average” odour per category, extracts were pooled according to the individual's parasitological status: (1) Plasmodium infection, no gametocytes (2) high-density P. falciparum gametocytes (3) parasite-free individuals, (4) Plasmodium infection, sub-microscopic P. falciparum gametocytes. Aliquots (400 μL) of extracts were mixed, then concentrated (to 60 μL) under a stream of nitrogen (charcoal-filtered). Glassware, charcoal filters and PTFE tubing were cleaned as before.

Experimental set-up

GC-EAG was conducted during the scotophase, using four- to eight- day-old, unfed female Anopheles coluzzii (N′gousso strain⁵⁶). Adults were maintained at 70% RH, with a 12 h light/dark cycle (scotophase 09:00-21:00) and access to 50% glucose solution. The order of testing blends was determined by a 5×5 Latin square (including control blend). The mosquito head was dissected, and the palps, proboscis, and half of the terminal (13^{th}) antennal flagellomere cut off. The indifferent electrode was inserted into the back of the head and the antennal tips guided into the recording electrode to complete the circuit (FIG. 3). Electrodes were hand-pulled glass tips inserted over silver wire (diameter 0.37 mm; Harvard Apparatus, Edenbridge, UK) and filled with Ringers' solution¹⁵. Gas chromatography (GC) was performed on a 7890A machine (Agilent Technologies®), with the following programme: oven temperature maintained at 40° C. for 0.5 minute, increased by 10° C. per minute to 230° C., then held for 20 minutes. The eluate was split to the FID detector and EAG interface at a ratio of 1:1. At the EAG interface, the eluate passed from the heated splitter column to a stream of charcoal filtered, humidified air (flow rate 400 mL/min). This airflow was directed over the antenna at a distance of 5 mm. The signal was amplified ×10,000 by the Intelligent Data Acquisition Controller-4, and signals were analysed using EAD 2000 software (both Syntech®, Hilversum, The Netherlands). Responses were signified by a depolarisation of sufficient amplitude. Peaks that elicited responses in more than 3, of the 6/7 total repetitions, were considered to be EAG-active.

Analysis of Odour Profiles by GC

Instruments used for GC analysis were 7890A, 6890N and HP6890 (Agilent Technologies, Stockport, UK). Each was fitted with a cool-on-column injector, flame ionization detector, used hydrogen carrier gas, and 1 μL injections were performed. All were fitted with an HP1 column, 50 m×0.32 mm, film thickness 0.52 μm, and the following programme was used: oven temperature maintained at 40° C. for 0.5 minutes, increased by 5° C. per minute to 150° C., held for 0.1 minute, raised by 10° C. per minute to 230° C., held for 40 minutes. Traces were analysed using the R package MALDIquant⁵⁷ (R version 3.3.0, 2016, The R Foundation for Statistical Computing©). In brief, raw x,y co-ordinates for GC traces were exported from Agilent ChemStation (C.01.04) and the y value (height, for 1 μL) multiplied by total extract to represent actual amount per sample (ng). Following baseline removal, traces were visually inspected for consistent differences between parasitological groupings. Compounds of interest (COI) were then compared quantitatively, by integrating peaks in ChemStation, and calculating retention index and amount relative to a standard series of n-alkanes (C7-C25), using Equation 1.

Equation 1. Retention index (RI) calculation

RI=100((log₁₀RtX−log₁₀Rtn)/(log₁₀Rtn+1−log₁₀Rtn))+100n
RtX=Retention time for compound of interest
Rtn=Retention time for alkane before compound of interest
Rtn+1=Retention time for alkane after compound of interest
n=number of carbons in alkane before compound of interest

Following statistical analysis (below), IAC were identified by gas chromatography-mass spectrometry (GC-MS), using either a Micromass Autospec Ultima (a magnetic sector mass spectrometer equipped with a Programmed Temperature Vaporising inlet (GL Sciences B.V., Eindhoven, The Netherlands) and Agilent 6890N GC), or a Mass Selective Detector (quad GC-MS). Peaks were compared with MS databases (National Institute of Standards and Technology, NIST). For confirmation of identification, authentic standards were injected onto two GC columns (HP1 and DB wax) simultaneously with samples containing those compounds. Standards were: heptanal (Sigma-Aldrich), octanal (Sigma-Aldrich), nonanal (Sigma-Aldrich), (E)-2-octenal (Acros Organics), (E)-2-decenal (SAFC), 2-octanone (Sigma-Aldrich). Identifications were considered certain when the resultant peak increased in height without increasing in width. Co-injections were conducted for all IAC.

Statistical Analysis

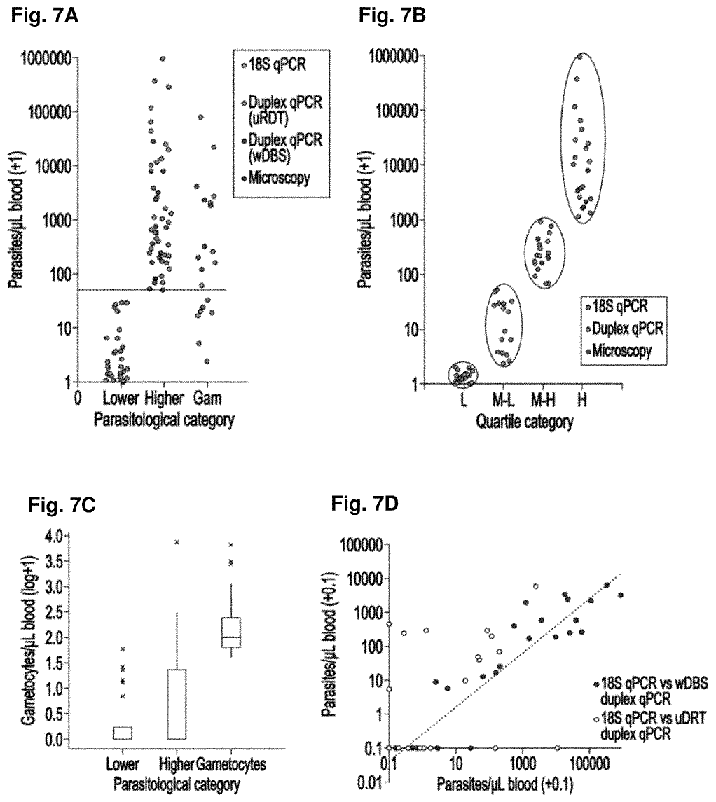

Any sample that had detectable parasite DNA at amounts greater than published limits of detection (LOD) for the assays (0.02 p/μL for 18S²³ and 5 p/μL for duplex qPCR²²) was considered positive, and those with DNA amounts beneath these thresholds were excluded. Only samples that were negative by all measures, including at least one molecular diagnostic measure, were taken to be negative, other than RDTs for which positivity was acceptable (on an assumption of positivity due to circulating HRP-2 protein)⁵⁸. Individuals with Plasmodium parasitaemia, but without microscopic gametocytes, were divided into higher and lower parasite density categories: ‘higher density’ with greater than 50 p/μL, and ‘lower density’ with between the LOD and 50 p/μL (FIG. 7A). Categorisation was informed using 18s qPCR, then duplex qPCR (wDBS>uRDT), then microscopy, according to assay result availability. Instances suggesting no parasites by 18S qPCR but with a robust parasite signal from one or more other measures were allocated to the appropriate positive category. For ‘quartile’ categories, ‘higher’ and ‘lower’ density samples (n=81) were then subdivided into quartiles according to density (FIG. 7B). Again, samples were allocated according to a hierarchy of procedures, in the order 18S qPCR >duplex qPCR >microscopy. Where 18S and/or duplex qPCR result was zero or missing but microscopy was positive, the film was re-read and that value assumed. Two samples with low parasite density by 18S but high and corresponding density by duplex qPCR and microscopy were allocated according to the two corresponding outcomes, and one further ‘lower density’ sample was excluded from ‘quartile’ analysis due to imprecise parasite density. Gametocyte densities per group, ‘total density’ categories, are given in FIG. 7C (measured QT-NASBA, where available), and the correlation between 18S qPCR and duplex qPCR by two templates in FIG. 7D.

The association between the production of COI (variate: percentage of total entrainment) and parasitological category was assessed by linear mixed models fitted using the method of residual maximum likelihood, REML. This modelling allowed for unequal sample sizes (per parasitological category) and repeated measures on the same individuals. We tested (F-tests) for the main effects of covariates (age, Hb, day of the year, weight) before the treatment (parasitological status) term, and for factors (sex, round) after the treatment term, in a forward selection, parallel-lines, regression analysis approach. Pairwise comparisons between groups of most biological interest were made using the LSD at the 5% level, and COI demonstrating significant (REML, LSD, 5%) differences between groups were termed ‘infection-associated compounds’. Data analysis was conducted using Genstat (2013, 16^{th} edition, VSN International, Hemel Hempstead, UK).

Behavioural Testing of Candidate Compounds

Testing IAC Individually

Six IAC (heptanal, octanal, nonanal, (E)-2-octenal, (E)-2-decenal and 2-octanone) were tested in a background of odour from the worn nylon socks of twelve parasite-free children (18S qPCR confirmed). Each sock pair was cut into twelve strips after removing the foot part, then 12 bundles were made, each containing a strip from each individual. Bundles were stored at −20° C. until, and between, experiments. IAC were positioned downwind and separated from sock bundles by a metal grid, ensuring no contact. Parasite-free odour (bundles) was tested with or without individual IAC (in 10 μL hexane on filter paper) and against the same but with hexane alone. For each IAC, a decimal dilution series was made (in hexane) and two/three concentrations chosen, to bracket the differential amount between significantly different groups (LSD 5%, REML), adjusted to represent 15 minutes of compound release (test duration).

Improving a Mosquito Lure

Next, we verified whether the IAC could improve a mosquito lure for monitoring or mass trapping of Anopheles. Heptanal, the most promising candidate from the above experiment, was tested as well as two blends: Plas 5 contained the IAC that were associated with parasitological positivity (nonanal, heptanal, octanal, (E)-2-decenal and (E)-2-octenal), and Plas 6 additionally contained the gametocyte-associated 2-octanone (FIG. 3). Each of Plas 5 and Plas 6 were made up in hexane by weighing the appropriate amounts of constituent compounds into a volumetric flask. Ratios were derived, and amounts of compounds were taken from predictions for compounds for parasitological groups with significantly increased quantity (LSD 5%, REML). Plas 5 and Plas 6 were tested with the synthetic lure MB5³⁶ at four concentrations, each decreasing by a factor of 10 from the 100% concentration by serial dilution. The stock compositions of Plas 5 and Plas 6 are shown in Table 1 below:

| | | Plas5 | Concentration | | Plas6 | Concentration |
|---|---|---|---|---|---|---|
| IAC | Plas5 % | proportion^{(d)} | (μg/ml) | Plas6 % | proportion^{(d)} | (μg/ml) |
| Nonanal^{(a)} | 16.36 | 1.00 | 47.60 | 16.36 | 1.00 | 47.60 |
| Octanal^{(a)} | 5.30 | 0.32 | 15.429 | 5.30 | 0.32 | 15.429 |
| Heptanal^{(a)} | 0.98 | 0.06 | 2.857 | 0.98 | 0.06 | 2.857 |
| (E)-2-octenal^{(b)} | 0.64 | 0.04 | 1.871 | 0.64 | 0.04 | 1.871 |
| (E)-2-decenal^{(b)} | 2.11 | 0.13 | 6.136 | 2.11 | 0.13 | 6.136 |
| 2-octanone^{(c)} | | | | 0.09 | 0.01 | 0.256 |

^{(a)}The predicted proportion of each compound found in the ‘higher’ density group (‘total density’ categorisation, REML) was used to generate values for octanal, nonanal and heptanal as the production of these compounds was upregulated in these groups
^{(b)}The predicted proportion found in the ‘positive’ group (‘positive vs. negative’ categorisation, REML) was used
^{(c)}The predicted proportion was taken from the ‘gametocyte’ group, ‘total density’ categorisation
^{(d)}Ratios were derived from these proportions, then all were normalised to the actual mean amount of nonanal found in the ‘higher’ density category (476 ng) in 100 min.

MB5 was presented on nylon strips as described in Menger et al. (2014)³⁶ (except the five compounds were incubated onto a single 5×26.5 cm nylon strip instead of five narrower strips). 10 μl of the Plas5 or Plas6 blend at 100% or serial dilution was pipetted onto a filter paper and added in the same olfactometer trap.

Assay

A triple chamber dual-port olfactometer⁵⁹ was used to test the preference of 30 five- to eight-day-old female, non blood-fed Anopheles coluzzii (Suokoko strain²¹, rearing procedures as published previously²¹) for parasite free odour or MB5, supplemented with IAC or IAC blends, against background odour alone (parasite-free odour or MB5). Mosquitoes were maintained in a release cage prior to testing (for 16-22 hours). Experiments took place during the last four hours of the scotophase under near-dark conditions (<1 lux). Mosquitoes were allowed to fly for 15 minutes, then those that had entered the traps with test/control odours were counted. Each IAC/concentration combination was tested eight-nine times on different days, and each Plas concentration 10-11 times on different days, rotating treatments between the left and right port of the olfactometer. Climatic data (R.H., temperature and air pressure) were recorded in the flight chambers and in the surrounding room.

Statistical Analyses

Generalized linear models (GLMs) were used to test the effect of odours (individual IAC/heptanal/Plas blends) on relative attractiveness (the proportion of mosquitoes selecting the test odour). GLMs were run as described above (statistical analysis, attractiveness of ‘infected odour’ (socks) by cage assays), testing parameters associated with the set-up as additional factors or covariates in the model, and retaining when significant (P<0.05, F-test). Sets of compounds were run in separate models. SPSS® was used for the analyses.

Plasmodium Manipulation of Odour Profile?

Parasite transmission often constitutes a population bottleneck: of the many parasites within one host, only a few are successfully transmitted to the next¹. Hence, parasites often evolve to exert influence over these transmission events. The malaria parasite Plasmodium would benefit from increasing its infected vertebrate host's attractiveness to susceptible Anopheles mosquito vectors, if this resulted in increased contact rates between the two hosts. Such changes in attractiveness in both animal²⁻⁶ and human⁷⁻⁹ malaria systems have previously been demonstrated. Changes in vertebrate host attractiveness in response to infection have also been documented in other vector-borne disease systems¹⁰⁻¹³, possibly indicating evolutionary convergence, which supports parasite manipulation underlying these phenomena¹. Body odour, comprising the volatile compounds emitted from the skin of vertebrates, is the most important cue used by Anopheles for host location¹⁴. It has been shown that differences in the composition of body odour are responsible for the variation in attractiveness to biting insects known to exist between people^{15,16}, and these differences may be influenced by body weight and/or surface area, hormones or genetic factors¹⁷⁻¹⁹. Human body odour can also be influenced by disease, including metabolic disorders, genetic disorders, and infections²⁰. A study of Plasmodium infection in mice found such changes in body odour to be associated with changes in attractiveness to mosquitoes⁵, and another found compositional changes in body odour during controlled human malaria infection (CHMI), with a variable effect on attractiveness²¹. While increased attractiveness of Plasmodium-infected individuals has been demonstrated in a malaria-endemic setting⁷, remarkably, no study has yet investigated the skin chemistry underlying this phenomenon. Given the crucial importance of body odour to mosquito host location, and the proposition that body odour can be altered during disease, here, we hypothesise that Plasmodium parasites manipulate the odour of infected humans, and that this influences attractiveness of humans to mosquitoes. To test this hypothesis, we first confirmed that asymptomatic children in Western Kenya were more attractive to mosquitoes when harbouring Plasmodium parasites, before comparing skin odour composition between Plasmodium-infected and parasite-free children from the same population. Using analytical chemistry, and the antennal and behavioural responses of Anopheles mosquitoes, we identified and established the role of Plasmodium infection-associated compounds in human body odour.

Attractiveness and Plasmodium Infection

To assess whether Plasmodium infection changes the attractiveness of human hosts to mosquitoes, we measured the behavioural response of Anopheles gambiae sensu stricto (s.s.) to foot odour of 5-12 year-old school children at two sampling time points. At time point one (T1), foot odour of asymptomatic Plasmodium falciparum-infected, and uninfected, children was collected on socks for 20 hours. For infected individuals, this occurred immediately after administration of the first dose of treatment with artemether-lumefantrine (AL), which is known to allow residual parasitaemia during this time period²².

Odour samples were collected in the same manner from the same children 21 days later, following confirmed parasite clearance (time point two, T2). Odour samples from participants with malaria parasites were categorized by those who harboured transmissible gametocyte stages (n=23), as identified either by microscopy or by the molecular diagnostic QT-NASBA that detects female gametocyte Pfs25 mRNA, or those with microscopically detected asexual stage parasites but no gametocytes (n=10). Samples were considered parasite-free (n=12) when no parasites were detected by microscopy and 18S qPCR²³. Anopheles gambiae s.s. mosquitoes were offered the choice of either T1 or T2 odour samples from the same child, in a dual choice cage assay (FIG. 2). The proportion of mosquitoes choosing the odours collected from children at T1 was significantly affected by parasitological status (GLM, F-test, P<0.001).

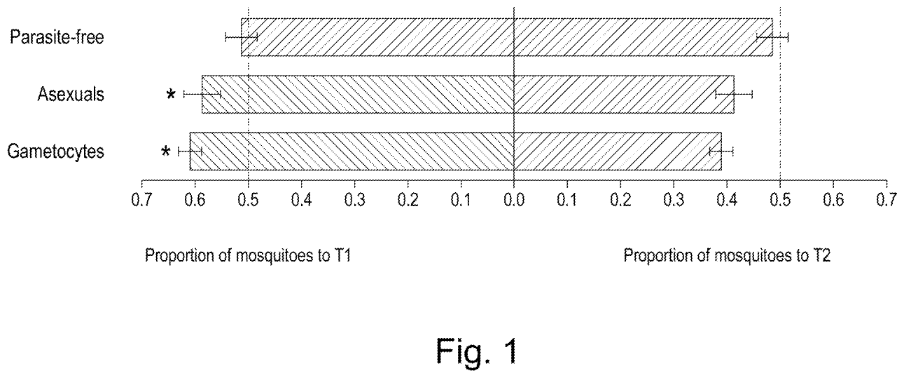

Mosquitoes were more attracted to odours collected at T1 from children harbouring asexual or gametocyte stage parasites relative to T2 odour samples (GLM, 95% confidence intervals (95 CI) 0.55-0.62 and 0.59-0.63 respectively, FIG. 1). Mosquitoes did not differentiate between T1 and T2 odour samples from parasite-free children, indicating that the difference observed between T1 and T2 odour was not an effect of sampling time point (GLM, 95 CI: 0.48-0.54, FIG. 1). This effect was independent of age, sex, tympanic (in-ear) temperature, or haemoglobin level at the first time point. These results indicate that infection with microscopically observable densities of either asexual stage parasites (median, 1340 [interquartile range, IQR: 480-2720] parasites/μL [p/μL]) or gametocytes (median, 80 [IQR: 40-680] p/μL) is associated with changes in odour profile that increase attraction to mosquitoes. This finding supports previous studies that demonstrate the heightened attractiveness of infected hosts, although here, by offering foot odour alone, we preclude the influence of other factors including breath. We did not observe the gametocyte-specific effect that was previously described⁷⁻⁹. To determine which chemicals in body odour are responsible for the observed differences in attractiveness, we repeat-sampled 56 Plasmodium-infected and parasite-free children from the same locality, using air entrainment to collect foot odour samples onto polymeric filters for further analysis.

Antennal Response to Malaria Odour

We analysed air entrainment odour extracts using coupled gas chromatography-electroantennography (GC-EAG)¹⁵. A change in the electric potential across the antenna resulting from stimulated neuropsychological activity, i.e. the EAG response, is caused during olfactory nerve cell response. This allows detection of compounds that are important to the mosquito (FIG. 3).

Point-of-care malaria diagnostics (rapid diagnostic test (RDT) and microscopy), used to inform odour sampling from asymptomatic individuals, were retrospectively confirmed using molecular diagnostics. Infected children were treated after odour sampling, and repeat sampling of all individuals was attempted one and three weeks later alongside repeat parasitological diagnoses (FIG. 3). Odour samples from individuals harbouring similar Plasmodium parasite stages or densities were extracted into solvent and mixed to create blends of ‘average’ odour with the following infection profiles: (1) Plasmodium infection, no gametocytes (2) Plasmodium infection, high-density gametocytes, and (3) parasite-free individuals,

A further group, (4) Plasmodium infection, sub-microscopic gametocytes, was included due to the frequency of sub-microscopic gametocytaemia in endemic infections. Plasmodium falciparum gametocyte densities were determined by Pfs25 mRNA QT-NASBA, while 18S qPCR and duplex qPCR were used to determine P. falciparum and Plasmodium densities respectively. Twenty-two analytes (Table 2) were found to elicit antennal response in Anopheles coluzzii (formerly the M-form of An. gambiae s.s. Giles), including the aldehydes heptanal, octanal and nonanal.

TABLE 2
Analytes (compounds eluting during gas chromatography, sometimes
at the same time) within odour samples that were found to induce
antennal response in Anopheles coluzzii. The
identification of analytes in bold font was subsequently
confirmed by co-injection with an authentic standard.

| RI^{(a)} | Tentative identification |
|---|---|
| 716 | Methyl cyclohexane |
| 750 | Unknown |
| 781 | Dimethyl sulfide |
| 795 | Solvent |
| 801 | Octane |
| 853 | 1-Hexanol (unsure identification), dimethyl sulfone |
| 880 | Heptanal |
| 889 | Solvent |
| 933 | Benzaldehyde |
| 958 | Phenol/1-octen-3-one |
| 982 | Octanal |
| 1014 | 2-Ethylhexanol or 2-octene |
| 1056 | Shoulder para cresol (4-methylphenol) and 3-octen-2-ol, |
| | peak octen-1-ol |
| 1084 | Nonanal |
| 1118 | Unknown |
| 1139 | 2-Ethyl benzaldehyde |
| 1150 | 4-Ethyl benzaldehyde |
| 1186 | Decanal |
| 1199 | Dodecane |
| 1239 | Ethylacetonphenone |
| 1389 | Unknown |
| 1428 | Geranylacetone |

^{(a)}Analytes identified by retention index (RI) were selected if eliciting >3 responses in one of the treatment groups (Plasmodium infection, no gametocytes, n = 6; Plasmodium infections, sub-microscopic P. falciparum gametocytes, n = 7; high-density P. falciparum gametocytes, n = 7; parasite-free individuals, n = 7; control, n = 7)

No EAG-active analytes were specific to any of the infection profiles (1-4), indicating that any Plasmodium-induced change in the compounds used by host-seeking Anopheles must manifest through variation in the relative amounts of compounds that are present in parasite-free individuals. This is in accordance with the ‘deceptive signalling’ hypothesis, whereby host cues already used by host-seeking insects are exaggerated, increasing the attractiveness of that vertebrate to biting insects, but when the blood-meal is in fact unfavourable to the insect³¹. If the disadvantages (e.g. reductions in fecundity²⁴⁻²⁶, shortened lifespan²⁷⁻²⁹) of taking an infected blood meal outweigh any advantages (e.g. reduced host defences², faster engorgement³⁰), the evolution of an infected-host avoidance phenotype might be expected. Anopheles may less easily select against an infected-host phenotype comprising ‘normal’ stimuli.

Plasmodium Infection-Associated Compounds (IAC)

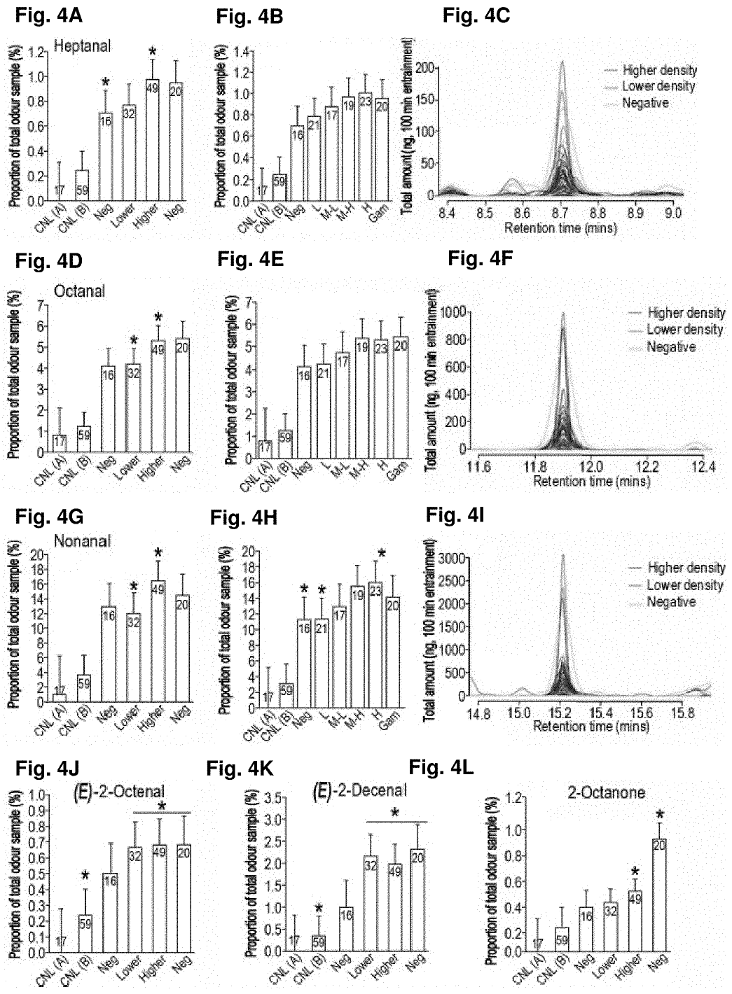

To investigate whether Plasmodium infection indeed results in quantitative changes in the production of volatile compounds, we compared the profiles of 117 foot, and 59 control (empty entrainment bag), odour samples. A total of 56 individuals participated in air entrainment odour sampling (FIG. 3), however, not all individuals were available at follow-up time points. Foot odour samples from Plasmodium-infected individuals were categorized by infection status: those from individuals with ‘higher’ (>50 p/μL, which approximates the microscopy limit of detection), and ‘lower’ (<50 p/μL), density infections, and those from individuals harbouring microscopic gametocytes (‘total density’ categorisation). As the prevalence of non-P. falciparum infections was low (5.05% [n=5] and 3.96% [n=4] for P. malariae and P. ovale spp. respectively at day 0, and eight of nine had concurrent P. falciparum parasites), we did not separate samples from individuals with non-falciparum infections. Our analysis revealed increases in the production of the aldehydes heptanal, octanal, nonanal, (E)-2-octenal, and (E)-2-decenal by infected individuals. Increases were broadly associated with infections of high parasite density, relative to either low density, or production by parasite-free individuals. High density infections were also correlated with the presence of gametocytes in this dataset (FIG. 7). Heptanal was produced in significantly greater amounts by individuals with higher parasite densities (>50 p/μL) relative to parasite-free individuals (REML, LSD, 5%, FIG. 4A/C). Octanal and nonanal were produced in significantly greater amounts by individuals with higher, relative to those with lower (<50 p/μL), density infections (REML, LSD, 5%, FIG. 44D/4F/4G/4I). To investigate further this seemingly density-dependent effect, we divided the ‘higher’ and ‘lower’ density individuals into quartiles, representing ‘low’, ‘medium-low’, ‘medium-high’, and ‘high’ density. We observed a clear correlation between increased production of heptanal, octanal and nonanal, and increased parasite density (FIGS. 4B/E/H). The difference in production of nonanal between ‘low’, or negative, and ‘high’ individuals was significant (REML, LSD, 5%, FIG. 4H). Relative to parasite-free individuals, there was a trend for all Plasmodium parasite-positive individuals to produce more of the aldehydes (E)-2-octenal and (E)-2-decenal (FIG. 4J/K), and for the latter, this difference was significant if individuals were categorized simply as Plasmodium-positive or parasite-free. Additionally, the ketone 2-octanone was found to be associated with the presence of microscopic gametocytes (REML, LSD, 5%, FIG. 4L).

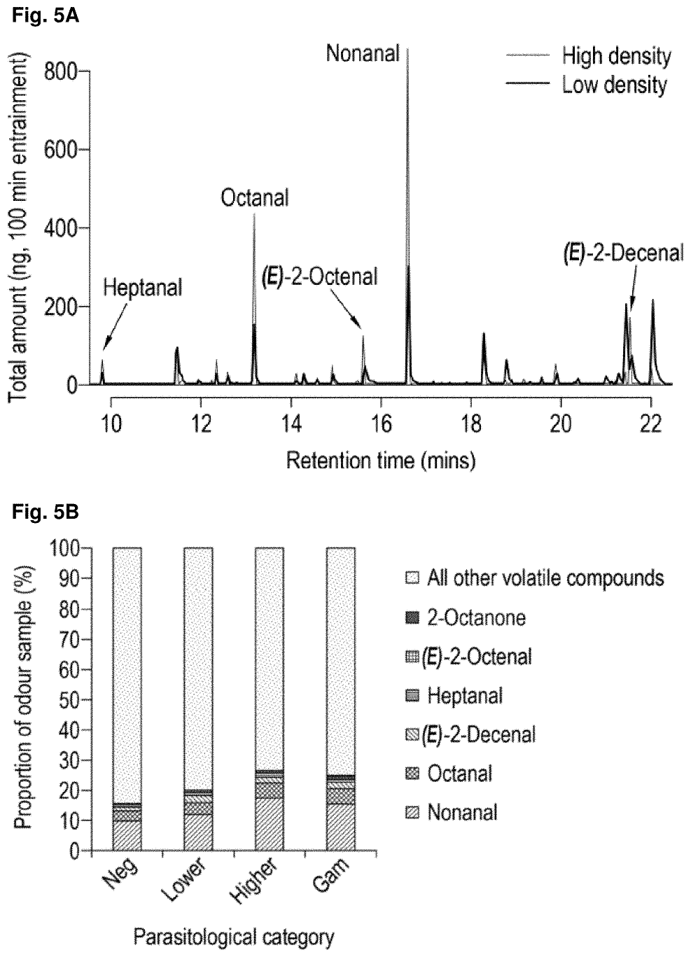

For all IAC, we found a quantitative relationship: the majority of individuals produced these compounds, but the quantity produced increased with Plasmodium infection. An average of 177 (standard error 5.23) analytes were captured per sample, and the IACs were disproportionately abundantly produced (FIG. 5), comprising on average 22.92% of the total odour profile across all 117 samples. When production was ranked relative to all other compounds sampled, nonanal had a median rank of one, octanal two and heptanal five. While specific IAC were produced in greater amounts by individuals harbouring parasites, an overall increase in volatile emissions from infected persons was not observed (REML, data not shown), contrary to findings in the mouse or CHMI system^{5,21}. Among the IAC, the antennal response, observed by GC-EAG to heptanal, octanal and nonanal, suggests that changes in the production of these compounds could affect mosquito behaviour.

Mosquito Response to IAC

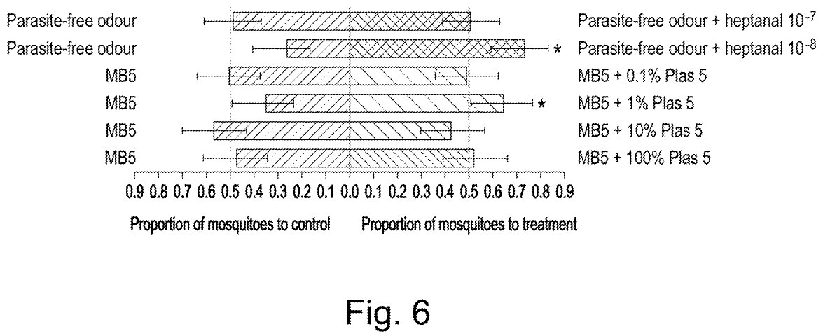

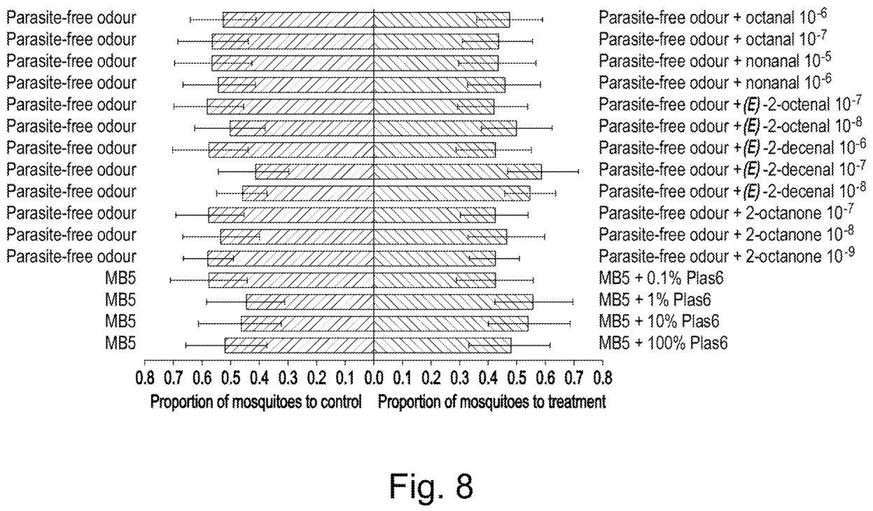

To determine whether the IAC were attractive to mosquitoes, and, therefore, likely to be responsible for the increased attractiveness observed in infected individuals, we tested all six IACs in behavioural bioassays with An. coluzzii. First, the odour of parasite-free children (worn socks) was supplemented with the IAC individually, and tested at a minimum of two concentrations each. Of these, adding 10 μL of heptanal at 10⁻⁸ g/mL to parasite-free odour significantly increased attractiveness, relative to parasite-free odour alone (GLM, 95 CI: 0.60-0.84, FIG. 6), while heptanal at 10⁻⁷ g/mL had no effect. The attractive concentration is approximately 1/10^{th} of the additional heptanal isolated in odour samples from individuals with ‘higher’ density Plasmodium infections, relative to negative individuals, over the corresponding time period. This suggests that elevated emission of heptanal, at specific concentrations, by parasitaemic children could contribute to their increased attractiveness to mosquitoes. Supplementing with octanal, nonanal, (E)-2-decenal, (E)-2-octenal or 2-octanone alone did not induce altered behavioural responses, despite the EAG-activity observed in response to octanal and nonanal (FIG. 8). We then tested whether the addition of heptanal to a current best-practice synthetic mosquito lure, MB5 (comprising ammonia, L-(+)-lactic acid, tetradecanoic acid, 3-methyl-1-butanol and butan-1-amine³⁶), might further increase attractiveness to mosquitoes. However, MB5 supplemented with heptanal was equally attractive as control MB5, at three concentrations (data not shown). This suggests that the attractiveness of heptanal observed with parasite-free odour was dependent on synergism with other volatile compounds naturally present, but absent from the synthetic MB5 blend. Because odour detection and response is highly contextual, this is not an unexpected outcome. To investigate further the behavioural role of IACs, but allowing for such synergistic effects between these compounds, we tested two blends with MB5: Plas 5 contained the aldehydes found to be associated with increased total parasite density (heptanal, octanal, nonanal, (E)-2-octenal and (E)-2-decenal), and Plas 6 additionally contained the ketone 2-octanone that was associated specifically with gametocytes. Each was tested at four concentrations. The Plas 5 blend enhanced attractiveness of MB5 (1% concentration, GLM, 95 CI: 0.51-0.77, FIG. 6). However, the Plas 6 blend was not found to increase attractiveness of MB5 at any concentration (FIG. 8), which suggests that the gametocyte-associated 2-octanone moderated the attractiveness of the Plas 5 aldehydes. Given the presence of small amounts of 2-octanone in parasite-free odour, however (FIG. 4I), which increases in attractiveness on addition of heptanal (FIG. 6), it appears that this repellency of 2-octanone is not observed in the context of natural human odour. In previous studies describing the increased attraction of gametocyte carriers, the odour tested included both body and breath⁷′⁹, leaving open the possibility that the gametocyte-specific attraction may have originated in the breath. Our behavioural tests show that supplementing parasite-free odour with heptanal increases attractiveness to mosquitoes. However, heptanal alone did not increase the attractiveness of a basic synthetic lure, while a blend of infection-associated aldehydes including heptanal (Plas 5) was attractive. Therefore, in both instances, the increased attraction was dependent on additive effects among the infection-associated aldehydes, which are naturally present in ‘parasite-free’ odour at lower concentrations (FIG. 4).

Aldehydes are found in the skin odour of various mammalian species³⁷, and have previously been determined to be among the chemicals used by haematophagous insects for host location³⁸. These oxygenated compounds can be synthesised when reactive oxygen species attack a lipid-dense membrane structure³⁹, i.e. lipid peroxidation, caused by oxidative stress. Oxidative stress is known to characterise malaria infection⁴⁰, occurring in the erythrocytes and liver. The probable presence of other infections in this cohort of children, including schistosomiasis, specifically associates the observed effect with Plasmodium infection itself, as a more general ‘scent of infection’ would likely be still present in the malaria-free individuals. Alternatively, or additionally, the aldehydes found here may have been produced directly by Plasmodium parasites: a recent publication found the aldehydes octanal, nonanal and decanal to be among volatile compounds emitted by red blood cell (RBC) cultures that had been supplemented by (E)-4-hydroxy-3-methyl-but-2-enyl pyrophosphate (HMBPP)⁴¹. HMBPP is a precursor in the 2-C-methyl-D-erythritol 4-phosphate (MEP) pathway, apparently used by Plasmodium for isoprenoid production, and it was suggested that HMBPP triggered enhanced release of these compounds from infected RBC, with a subsequent impact on mosquito attraction. Additionally, terpenes were isolated from HMBPP RBC, and another study also isolated terpenes above Plasmodium infected RBC cultures⁴². Although the MEP pathway is a possible source of terpenes via isoprenoid production in infected RBC⁴², the source of terpenes in HMBPP RBC remains unknown⁴¹. We did not find an association between Plasmodium infection and the emission of terpenes from the skin, corroborating earlier findings in Plasmodium-infected mice⁵. It should be emphasised that laboratory-based studies of the volatile compounds isolated above iRBC cultures do not characterise the human body odour used by mosquitoes during host location. As such, they do not fully capture the complex biological and biochemical host-parasite interactions that occur in natural Plasmodium infections. In our study, the production of aldehydes was increased in individuals with Plasmodium infection. The extent to which parasite-specific release of aldehydes from iRBC would contribute to a profound and systemic increase in aldehyde production, caused by malaria-induced oxidative stress, remains unexplored. Finally, it is important to note that while the lipid peroxidation pathway for aldehyde production is well-established, the skin microbiota are also known to produce aldehydes. This is particularly relevant to our study, as odour samples were taken from the feet. Feet harbour skin microflora that produce volatiles that are attractive to mosquitoes⁴³, and differences in microflora have been associated with differences in attractiveness¹⁶.

### Example 2

Table 3 below shows the results of analysis of sampled foot odour from Plasmodium-infected and non-infected individuals. The percentage composition of foot odours for the various volatile compounds are shown, as well as the mean amounts, in ng, relative to nonanal. The amounts of compounds were collected in 100 minutes sampling from the foot only.

| | Percentage of | | Mean amount found | |
|---|---|---|---|---|
| | odour sample | | relative to nonanal, ng* | |
| Individual: | Negative | Infected | Negative | Infected |
| Hexanal | 1.699 | 2.786 | 64.234 | 70.326 |
| Heptanal | 0.610 | 1.077 | 23.050 | 29.750 |
| 1-Octen-3-one | 0.402 | 0.753 | 15.216 | 19.014 |
| 2-Octanone | 0.032 | 0.051 | 1.229 | 1.410 |
| Octanal | 3.157 | 5.224 | 119.392 | 144.275 |
| (E )-2-Octenal | 0.455 | 0.805 | 17.208 | 20.348 |
| Nonanal | 9.837 | 17.237 | 371.977 | 476.035 |
| (E )-2-Decenal | 1.176 | 2.275 | 44.484 | 57.509 |

Discussion

We demonstrated that elevated production of specific aldehydes in skin odour is associated with increased attractiveness to mosquitoes in Plasmodium-infected people. We found that odour from all P. falciparum-infected individuals was more attractive than that of parasite-free individuals, and the increased production of IACs was correlated with total parasite density. The association between P. falciparum asexual parasite biomass and gametocyte density is generally positive⁴⁴⁻⁴⁶, and we also observed this in our study. The application of identifying such infection-associated compounds, with their demonstrated impact on mosquito behaviour, are far-reaching: we better understand parasite-vector-host transmission events, and their over-dispersed nature in human populations. These compounds are expected to permit further improvement of already highly functional lures for trapping malaria mosquitoes, or to serve as biomarkers for malaria, providing a basis for novel and non-invasive diagnostic tools.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Example 3

A synthetic lure composed of the Mbita blend (MB5) and the Plas 5 blend has been found to attract Anopheles mosquitoes in a field trial undertaken in Bubaque, Bijagos archipelago. The synthetic lure was used to bait Centre of Disease Control (CDC) Light traps at three concentrations (0.1% (v/v), 1% (v/v) and 10% (v/v) Plas 5 with MB5. As well as successfully attracting Anopheles mosquitoes, mosquitoes of the genera Aedes and Culex were also attracted to the lures. A total of 2134 mosquitoes were caught over 25 nights across the five treatments. Of this, 206 (9.7%) consisted of female anophelines. Traps baited with MB5+1% Plas 5 had the highest Anopheles females capture rate (n=13.4) and this treatment caught the most substantial number consistently. The combination of MB5+10% Plas 5 had the highest absolute mosquito capture rate (n=106). FIGS. 9 and 10 visualise the mosquito abundances with treatment. Using a 5×5 Latin Square design, the control system demonstrated the ability to attract Anopheles and Culex mosquitoes. No Aedes mosquitoes (FIG. 10) were caught using the control. The control (FIG. 9) demonstrated the lowest relative proportion of Anopheles females caught.

For each genera-sex group (FIG. 10) increased concentration of MB5+Plas 5 combination produced different patterns of trap catches. The abundance of Culex males increased with increased concentration, whereas the abundance of Anopheles females peaked at MB5+1% Plas 5. In contrast, the gradual increase of Cu/ex females dipped at MB5+1% Plas 5. Across all genera groups, there was a significant effect of treatment on the total trap catch. The greatest significant effect was across both Anopheles females (p<0.001) and Culex females (p<0.001). Anopheles males (p<0.05) Culex males p(<0.05) and Aedes females (p<0.05) also had significant differences between treatments, to lesser effects. No Aedes males were caught.

A greater number of Anopheles mosquitoes (FIG. 10) were caught using the novel synthetic lure, across all concentrations than compared to the controls. The trap catch index (see table 4 below) was determined for each treatment for the absolute number of mosquitoes and Anopheles females. The trap-catch index of female Anopheles caught using all concentrations of the novel synthetic lure was greater than that of MB5 alone. Trap catch indexes of 2.25, 3.35 and 2.5 with increasing concentration of Plas 5 compared to the control. The Anopheles trap catch index of MB5 was only 1.2 greater than that of the control.

TABLE 4
Trap catches indexes comparing the absolute trap catches and the total Anopheles
mosquitoes across treatments.

| | Absolute | Anopheles | Absolute | Anopheles | Absolute | Anopheles | Absolute | Anopheles |
|---|---|---|---|---|---|---|---|---|
| Hexane | — | — | — | — | — | — | — | — |
| MB5 | 1.937759336 | 1.2 | — | — | — | — | — | — |
| 0.1% Plas 5 | 2.012448133 | 2.25 | 1.038544 | 1.875 | — | — | — | — |
| 1% Plas 5 | 1.705394191 | 3.35 | 0.880086 | 2.791666667 | 0.847423 | 1.48888889 | — | — |
| 10% Plas 5 | 2.199170124 | 2.5 | 1.134904 | 2.083333333 | 1.092784 | 1.11111111 | 1.289538 | 0.746268657 |

Treatment 4, MB5+1% Plas 5 was shown to be the concentration that was the most successful in attracting Anopheles mosquitoes. In addition to the highest capture rate as mentioned previously, the trap catch index was also the greatest (3.35). Furthermore, the MB5+1% Plas 5 catch indexes (table 4) were higher than 1 against both the hexane control and MB5, as well as to MB5+0.1% Plas 5 and MB5+10% Plas 5.

Treatment Effects

There was no correlation between the environmental variables of temperature, wind speed, humidity or rainfall present with the trap catches. Therefore, the model effects of these parameters were not included as covariates in the models created.

Anopheles Females

Tukey post-hoc results (see table 5 below) confirmed the significant effect of treatment on the relative abundance of Anopheles females. There was no significant difference between the control and MB5 treatments, p=0.77. However, there were significant differences between the control and 0.1% Plas 5 (p<0.05) and 10% Plas 5 (p<0.01) with the significant difference between the control and 1% Plas 5 being the greatest (p<0.001). Comparing the combined MB5+Plas 5 baits to MB5 alone, there was only a significant effect between 1% Plas 5 and MB5 (p<0.01). There were no other significant differences between treatments.

TABLE 5
Multiple comparisons of treatments to determine the direction
of the odour treatment effect on Anopheles:

| | Estimate | Standard | | |
|---|---|---|---|---|
| Treatment | of | error of | Z- | |
| comparison | coefficient | coefficient | value | P-value |
| MB5 − Hexane | 0.33647 | 0.29275 | 1.149 | 0.77573 |
| MB5 + 0.1% | 0.81093 | 0.26872 | 3.018 | 0.02080 |
| Plas 5 − Hexane | | | | |
| MB5 + 1% | 1..16315 | 0.25615 | 4.541 | <0.001 |
| Plas 5 − Hexane | | | | |
| MB5 + 10% | 0.89609 | 0.26532 | 3.377 | 0.00637 |
| Plas 5 − Hexane | | | | |
| MB5 + 0.1% | 0.47446 | 0.24070 | 1.971 | 0.27466 |
| Plas 5 − MB5 | | | | |
| MB5 + 1% | 0.82668 | 0.22658 | 3.648 | 0.00238 ** |
| Plas 5 − MB5 | | | | |
| MB5 + 10% | 0.55962 | 0.23690 | 2.362 | 0.12251 |
| Plas 5 − MB5 | | | | |
| MB5 + 1% | 0.35222 | 0.19454 | 1.810 | 0.36123 |
| Plas 5 − MB5 + | | | | |
| 0.1% Plas 5 | | | | |
| MB5 + 10% | 0.08516 | 0.20647 | 0.412 | 0.99377 |
| Plas 5 − MB5 + | | | | |
| 0.1% Plas 5 | | | | |
| MB5 + 10% | −0.26706 | 0.18982 | −1.407 | 0.61728 |
| Plas 5 − MB5 + | | | | |
| 1% Plas 5 | | | | |

For Culex females (see table 6 below), Poisson regression models revealed that there was a significant difference between all treatments and the control with p<0.001. Treatment 3 and treatment 5 had a significant difference of p<0.005 and p<0.001 respectively. Therefore, treatment 4 had a significantly lower abundance than the other MB5+Plas 5 treatments. None of the treatments was significantly different to MB5 alone.

TABLE 6
Multiple comparisons of treatments to determine the direction
of the odour treatment effect on Culex females:

| | | Standard | | |
|---|---|---|---|---|
| Treatment | Estimate | error of | | |
| comparison | coefficient | coefficient | Z-value | P-value |
| MB5 − Hexane | 0.75803 | 0.09058 | 8.369 | <0.001 |
| MB5 + 0.1% | 0.83606 | 0.08948 | 9.343 | <0.001 |
| Plas 5 − Hexane | | | | |
| MB5 + 1% | 0 0.57781 | 0.09339 | 6.187 | <0.001 |
| Plas 5 − Hexane | | | | |
| MB5 + 10% | 0. 87872 | 0.08892 | 9.882 | <0.001 |
| Plas 5 − Hexane | | | | |
| MB5 + 0.1% | 0.07803 | 0.07099 | 1.099 | 0.80463 |
| Plas 5 − MB5 | | | | |
| MB5 + 1% | −0.18023 | 0.07585 | −2.376 | 0.11983 |
| Plas 5 − MB5 | | | | |
| MB5 + 10% | 0.12069 | 0.07027 | 1.717 | 0.41917 |
| Plas 5 − MB5 | | | | |
| MB5 + 1% | −0.25826 | 0.07454 | −3.465 | 0.00471 |
| Plas 5 − MB5 + | | | | |
| 0.1% Plas 5 | | | | |
| MB5 + 10% | 0.04266 | 0.06886 | 0.620 | 0.97158 |
| Plas 5 − MB5 + | | | | |
| 0.1% Plas 5 | | | | |
| MB5 + 10% | 0.30092 | 0.07386 | 4.074 | <0.001 |
| Plas 5 − MB5 + | | | | |
| 1% Plas 5 | | | | |

In Culex males (see table 7 below), there were less significant differences in abundances between treatments. Only treatment 5 was significantly different, to the control with p<0.05 and to MB5, p<0.05. there was no significant difference between MB5+Plas 5 treatments or for the treatments against the control.

TABLE 7
Multiple comparisons of treatments to determine the direction
of the odour treatment effect on Culex males.

| | | Standard | | |
|---|---|---|---|---|
| | Estimate | error of | | |
| Treatment comparison | coefficient | coefficient | Z-value | P-value |
| MB5 −Hexane | 0.07696 | 0.27754 | 0.277 | 0.9987 |
| MB5 + 0.1% Plas 5 − Hexane | 0.27763 | 0.26513 | 1.047 | 0.8318 |
| MB5 + 1% Plas 5 − Hexane | 0.39204 | 0.25887 | 1.514 | 0.5505 |
| MB5 + 10% Plas 5 − Hexane | 0.73237 | 0.24335 | 3.010 | 0.0217 * |
| MB5 + 0.1% Plas 5 − MB5 | 0.20067 | 0.25950 | 0.773 | 0.9377 |
| MB5 + 1% Plas 5 − MB5 | 0.31508 | 0.25311 | 1.245 | 0.7229 |
| MB5 + 10% Plas 5 − MB5 | 0.65541 | 0.23721 | 2.763 | 0.0450 * |
| MB5 + 1% Plas 5 − MB5 + | 0.11441 | 0.23944 | 0.478 | 0.9893 |
| 0.1% Plas 5 | | | | |
| MB5 + 10% Plas 5 − MB5 + | 0.45474 | 0.22256 | 2.043 | 0.2433 |
| 0.1% Plas 5 | | | | |
| MB5 + 10% Plas 5 − MB5 + | 0.34033 | 0.21508 | 1.582 | 0.5062 |
| 1% Plas 5 | | | | |

Molecular Identification

Anopheles:

Overall 206 female Anopheles were caught across traps of the five treatments. PCR results of the trap catches (FIG. 11) revealed species in the Anopheles gambiae complex: Anopheles gambiae s.s, An. Melas, An. Coluzzii and M/S hybrids. Of this, the largest species in abundance, 52% (n=108) was Anopheles melas. 7.8% (n=16) were found to be Anopheles gambiae colluzzii (M) and 15% (n=31) were Anopheles gambiae gambiae (S). A final 15% (n=31) were M/S hybrids. The remaining 10% of Anopheles were unable to be read on the gel screen. The lack could be due to errors during PCR amplification. Alternatively, as the primers used in the Scott et al PCR are specific to species in the Anopheles gambiae s.l complex it could indicate the presence of Anopheles not within Anopheles gambiae s.l complex⁴⁸. The abundances of each species relative to the treatment it was caught using is expressed in table 8 below. However, molecular speciation using endpoint

TABLE 8
The abundance of each species Anopheles gambiae s.l complex with treatment.

| | Treatment | | | | | | | | | |
|---|---|---|---|---|---|---|---|---|---|---|
| | Hexane | | MB5 | | 0.1% Plas 5 | | 1% Plas 5 | | 10% Plas 5 | |
| Anopheles | n | % | n | % | n | % | n | % | n | % |
| An. melas | 9 | 4.37 | 18 | 8.74 | 26 | 12.62 | 34 | 16.50 | 21 | 10.19 |
| An. Coluzzii (M) | 2 | 0.97 | 1 | 0.49 | 2 | 0.97 | 5 | 2.43 | 6 | 2.91 |
| An. gambiae (S) | 5 | 2.43 | 4 | 1.94 | 3 | 1.46 | 11 | 5.34 | 8 | 3.88 |
| An. Gambiae | 2 | 0.97 | 2 | 0.97 | 6 | 2.91 | 10 | 4.85 | 11 | 5.34 |
| hybrid (M/S) | | | | | | | | | | |
| Total | 12 | | 25 | | 37 | | 60 | | 46 | |

PCR determined that there was no significant effect of treatment on the species of Anopheles caught, p=0.1718. This is illustrated in table 7.

Following the CSP ELISA, the sporozoite rate of infective anophelines was calculated as 0.97%. Of the Anopheles females with sporozoites detected, one was An. gambiae (S) under 1% Plas 5 treatment. The other was Anopheles melas and was under the hexane control treatment. The sample size of Anopheles was not large enough to be the basis for an accurate depiction of local sporozoite rates.

Culex:

Morphologically, only two of the ten morphological groups were able to be identified to species. These were Culex quinquefasciatus and Culex tritaenoiorhynchus. The remaining species are possible of non-medical importance as they could not be identified with the medical mosquito keys.

The results of the trapping experiment indicate an optimisation of attractant of Plas5 at about 1% (v/v) in MB5 in order to trap proportionally more Anopheles female mosquitoes than the MB5 blend alone. Plas5 may be used, for example in the range 0.5%-5% (v/v) in order to potentiate existing attractant blends to increase their attractiveness for Anopheles females.

### REFERENCES

1. Poulin, R. Chapter 5—Parasite Manipulation of Host Behavior: An Update and Frequently Asked Questions. in Advances in the Study of Behavior 41, 151-186 (2010).
2. Day, J. F. & Edman, J. D. Malaria renders mice susceptible to mosquito feeding when gametocytes are most infective. J. Parasitol. 69, 163-70 (1983).
3. Coleman, R. E., Edman, J. D. & Semprevivo, L. H. Interactions between malaria (Plasmodium yoelii) and leishmaniasis (Leishmania mexicana amazonensis): effect of concomitant infection on host activity, host body temperature, and vector engorgement success. J. Med. Entomol. 25, 467-71 (1988).
4. Ferguson, H. M., Rivero, A. & Read, A. F. The influence of malaria parasite genetic diversity and anaemia on mosquito feeding and fecundity. Parasitology 127, 9-19 (2003).
5. De Moraes, C. M., Stanczyk, N. M., Betz, H. S., Pulido, H., Sim, D. G., et al. Malaria-induced changes in host odors enhance mosquito attraction. Proc. Natl. Acad. Sci. U.S.A. 111(30), 11079-11084 (2014)
6. Cornet, S., Nicot, A., Rivero, A. & Gandon, S. Malaria infection increases bird attractiveness to uninfected mosquitoes. Ecol. Lett. 16, 323-9 (2012).
7. Busula, A. O., Bousema, T., Mweresa, C. K., Masiga, D., Logan, J. G., et al. Gametocytaemia increases attractiveness of Plasmodium falciparum-infected Kenyan children to Anopheles gambiae mosquitoes. J. Infect. Dis. 216(3):291-295 (2017)
8. Batista, E. P., Costa, E. F. & Silva, A. a. Anopheles darlingi (Diptera: Culicidae) displays increased attractiveness to infected individuals with Plasmodium vivax gametocytes. Parasit. Vectors 7, 251 (2014).
9. Lacroix, R., Mukabana, W. R., Gouagna, L. C. & Koella, J. C. Malaria infection increases attractiveness of humans to mosquitoes. PLoS Biol. 3, e298 (2005).
10. Turell, M. J., Bailey, C. L. & Rossi, A. A. Increased mosquito feeding on rift valley fever virus-infected lambs. 33, 1232-1238 (1984).
11. Coleman, R. E. & Edman, J. D. Feeding-site selection of Lutzomyia longipalpis (Diptera: Psychodidae) on mice infected with Leishmania mexicana amazonensis. J. Med. Entomol. 25, 229-33 (1988).
12. Baylis, M. & Nambiro, C. O. The effect of cattle infection by Trypanosoma congolense on the attraction, and feeding success, of the tsetse fly Glossina pallidipes. Parasitology 106 (Pt 4, 357-61 (1993).
13. O'Shea, B., Rebollar-Tellez, E., Ward, R. D., Hamilton, J. G. C., el Naiem, D., et al. Enhanced sandfly attraction to Leishmania-infected hosts. Trans. R. Soc. Trop. Med. Hyg. 96, 117-8 (2002).
14. Takken, W. & Knols, B. G. Odor-mediated behavior of Afrotropical malaria mosquitoes. Annu. Rev. Entomol. 44, 131-57 (1999).
15. Logan, J. G., Birkett, M. A., Clark, S. J., Powers, S., Seal, N. J., et al. Identification of human-derived volatile chemicals that interfere with attraction of Aedes aegypti mosquitoes. J Chem Ecol 34, 308-322 (2008).
16. Verhulst, N. O., Qiu, Y. T., Beijleveld, H., Maliepaard, C., Knights, D., et al. Composition of human skin microbiota affects attractiveness to malaria mosquitoes. PLoS ONE [Electronic Resour. 6, e28991 (2011).
17. Muirhead-Thomson, R. C. The distribution of anopheline mosquito bites among different age groups; a new factor in malaria epidemiology. Br. Med. J. 1, 1114-7 (1951).
18. Gilbert, I. H., Gouck, H. K. & Smith, N. Attractiveness of Men and Women to Aedes aegypti and Relative Protection Time Obtained with Deet. Florida Entomol. 49, 53 (1966).
19. Fernández-Grandon, G. M., Gezan, S. a., Armour, J. a. L., Pickett, J. a. & Logan, J. G. Heritability of Attractiveness to Mosquitoes. PLoS One 10, e0122716 (2015).
20. Prugnolle, F., Lefèvre, T., Renaud, F., Møller, A. P., Missé, D., et al. Infection and body odours: Evolutionary and medical perspectives. Infection, Genetics and Evolution 9, 1006-1009 (2009).
21. de Boer, J. G., Robinson, A., Powers, S. J., Burgers, S. L. G. E., Caulfield, J. C., et al. Odours of Plasmodium falciparum-infected participants influence mosquito-host interactions. Sci. Rep. 7(1):9283 (2017).
22. Beshir, K. B., Hallett, R. L., Eziefula, A. C., Bailey, R., Watson, J., et al. Measuring the efficacy of anti-malarial drugs in vivo: quantitative PCR measurement of parasite clearance. Malar. J. 9, 312 (2010).
23. Hermsen, C. C., Telgt, D. S., Linders, E. H., van de Locht, L. a, Eling, W. M., et al. Detection of Plasmodium falciparum malaria parasites in vivo by real-time quantitative PCR. Mol. Biochem. Parasitol. 118, 247-251 (2001).
24. Hacker, C. The differential effect of Plasmodium gallinacium on the fecundity of several strains of Aedes aegypti. J. Invertebr. Pathol. 18, 373-377 (1971).
25. Freier, J. E. & Friedman, S. Effect of host infection with Plasmodium gallinaceum on the reproductive capacity of Aedes aegypti. J. Invertebr. Pathol. 28, 161-6 (1976).
26. Vézilier, J., Nicot, A., Gandon, S. & Rivero, A. Plasmodium infection decreases fecundity and increases survival of mosquitoes. Proc. R. Soc. London B Biol. Sci. (2012).
27. Anderson, R. A., Knols, B. G. & Koella, J. C. Plasmodium falciparum sporozoites increase feeding-associated mortality of their mosquito hosts Anopheles gambiae s.l. Parasitology 120 (Pt 4), 329-33 (2000).
28. Ferguson, H. M. & Read, A. F. Genetic and environmental determinants of malaria parasite virulence in mosquitoes. Proceedings. Biol. Sci. 269, 1217-24 (2002).
29. Dawes, E. J., Churcher, T. S., Zhuang, S., Sinden, R. E. & Basãñez, M.-G. Anopheles mortality is both age- and Plasmodium-density dependent: implications for malaria transmission. Malar. J. 8, 228 (2009).
30. Rossignol, P. A., Ribeiro, J. M. C., Jungery, M. & Spielman, A. Enhanced mosquito blood-finding success on parasitemic hosts: Evidence for vector-parasite mutualism. Proc. Natl. Acad. Sci. U.S.A. 82, 7725-7727 (1985).
31. Mauck, K. E., De Moraes, C. M. & Mescher, M. C. Deceptive chemical signals induced by a plant virus attract insect vectors to inferior hosts. Proc. Natl. Acad. Sci. U.S.A. 107, 3600-5 (2010).
32. Bernier, U. R., Booth, M. M. & Yost, R. A. Analysis of human skin emanations by gas chromatography/mass spectrometry. 1. Thermal desorption of attractants for the yellow fever mosquito (Aedes aegypti) from handled glass beads. Anal. Chem. 3, 1-7 (1999).
33. Curran, A. M., Rabin, S. I., Prada, P. A. & Furton, K. G. Comparison of the volatile organic compounds present in human odor using SPME-GC/MS. J. Chem. Ecol. 31, 1607-19 (2005).
34. Penn, D. J., Oberzaucher, E., Grammer, K., Fischer, G., Soini, H. A., et al. Individual and gender fingerprints in human body odour. J. R. Soc. Interface 4, 331-40 (2007).
35. Dormont, L., Bessière, J.-M. & Cohuet, A. Human skin volatiles: a review. J. Chem. Ecol. 39, 569-78 (2013).
36. Menger, D. J., Van Loon, J. J. A. & Takken, W. Assessing the efficacy of candidate mosquito repellents against the background of an attractive source that mimics a human host. Med. Vet. Entomol. 28, 407-13 (2014).
37. Jaleta, K. T., Hill, S. R., Birgersson, G., Tekie, H. & Ignell, R. Chicken volatiles repel host-seeking malaria mosquitoes. Malar. J. 15, 354 (2016).
38. Puri, S. N., Mendki, M. J., Sukumaran, D., Ganesan, K., Prakash, S., et al. Electroantennogram and Behavioral Responses of Culex quinquefasciatus (Diptera: Culicidae) Females to Chemicals Found in Human Skin Emanations. J. Med. Entomol. 43, 207-213 (2006).
39. Fuchs, P., Loeseken, C., Schubert, J. K. & Miekisch, W. Breath gas aldehydes as biomarkers of lung cancer. Int. J. Cancer 126, 2663-2670 (2010).
40. Becker, K., Tilley, L., Vennerstrom, J. L., Roberts, D., Rogerson, S., et al. Oxidative stress in malaria parasite-infected erythrocytes: Host-parasite interactions. Int. J. Parasitol. 34, 163-189 (2004).
41. Emami, S. N., Emami, S. N., Lindberg, B. G., Hua, S., Hill, S., et al. A key malaria metabolite modulates vector blood seeking, feeding, and susceptibility to infection. Science (80-.). 4563, 1-9 (2017).
42. Kelly, M., Su, C.-Y., Schaber, C., Crowley, J. R., Hsu, F.-F., et al. Malaria parasites produce volatile mosquito attractants. MBio 6, e00235-15-(2015).
43. Verhulst, N. O., Beijleveld, H., Knols, B. G., Takken, W., Schraa, G., et al. Cultured skin microbiota attracts malaria mosquitoes. Malar. J. 8, 302 (2009).
44. Bousema, T. & Drakeley, C. Epidemiology and infectivity of Plasmodium falciparum and Plasmodium vivax gametocytes in relation to malaria control and elimination. Clin. Microbiol. Rev. 24, 377-410 (2011).
45. Koepfli, C., Robinson, L. J., Rarau, P., Salib, M., Sambale, N., et al. Blood-Stage Parasitaemia and Age Determine Plasmodium falciparum and P. vivax Gametocytaemia in Papua New Guinea. PLoS One 10, e0126747 (2015).
46. Slater, H. C., Ross, A., Ouedraogo, A. L., White, L. J., Nguon, C., et al. Assessing the impact of next-generation rapid diagnostic tests on Plasmodium falciparum malaria elimination strategies. Nature 528, S94-S101 (2015).
47. Okal, M. N., Francis, B., Herrera-Varela, M., Fillinger, U. & Lindsay, S. W. Water vapour is a pre-oviposition attractant for the malaria vector Anopheles gambiae sensu stricto. Malar. J. 12, 365 (2013).
48. WHO. Guidelines for testing mosquito adulticides for indoor residual spraying and treatment of mosquito nets. (2006). doi:Ref: WHO/CDS/NTD/WHOPES/GCDPP
49. Busula, A. O., Takken, W., Loy, D. E., Hahn, B. H., Mukabana, W. R., et al. Mosquito host preferences affect their response to synthetic and natural odour blends. Malar. J. 14, 133 (2015).
50. Dicko, A., Brown, J. M., Diawara, H., Baber, I., Mahamar, A., et al. Primaquine to reduce transmission of Plasmodium falciparum malaria in Mali: a single-blind, dose-ranging, adaptive randomised phase 2 trial. Lancet Infect. Dis. 16, 674-684 (2016).
51. Schneider, P., Schoone, G., Schallig, H., Verhage, D., Telgt, D., et al. Quantification of Plasmodium falciparum gametocytes in differential stages of development by quantitative nucleic acid sequence-based amplification. Mol. Biochem. Parasitol. 137, 35-41 (2004).
52. Cnops, L., Boderie, M., Gillet, P., Van Esbroeck, M. & Jacobs, J. Rapid diagnostic tests as a source of DNA for Plasmodium species-specific real-time PCR. Malar. J. 10, 67 (2011).
53. Robinson, A., Muwanguzi, J., Busula, A. O., Masiga, D., Powers, S., et al. Quantification of bloodstream parasitaemia by duplex qPCR using a rapid diagnostic test template. (In prep)
54. Snounou, G., Viriyakosol, S., Xin Ping Zhu, Jarra, W., Pinheiro, L., et al. High sensitivity of detection of human malaria parasites by the use of nested polymerase chain reaction. Mol. Biochem. Parasitol. 61, 315-320 (1993).
55. Oguike, M. C., Betson, M., Burke, M., Nolder, D., Stothard, J. R., et al. Plasmodium ovale curtisi and Plasmodium ovale wallikeri circulate simultaneously in African communities. Int. J. Parasitol. 41, 677-683 (2011).
56. Habtewold, T., Duchateau, L. & Christophides, G. K. Flow cytometry analysis of the microbiota associated with the midguts of vector mosquitoes. Parasit. Vectors 9, 167 (2016).
57. Gibb, S. MALDIquant: Quantitative Analysis of Mass Spectrometry Data. 1-16 (2014).
58. Abba, K., Deeks, J. J., Olliiaro, P., Naing, C.-M., Jackson, S. M., et al. Rapid diagnostic tests for diagnosing uncomplicated P. falciparum malaria in endemic countries. Cochrane database Syst. Rev. CD008122 (2011). doi:10.1002/14651858.CD008122.pub2
59. Verhulst, N. O., Weldegergis, B. T., Menger, D. & Takken, W. Attractiveness of volatiles from different body parts to the malaria mosquito Anopheles coluzzii is affected by deodorant compounds. Sci. Rep. 6, 27141 (2016).

1. A mosquito attractant composition comprising heptanal, octanal, nonanal, (E)-2-octenal and (E)-2-decenal.

2. The mosquito attractant composition of claim 1, comprising the following ratios of heptanal, octanal, nonanal, (E)-2-octenal and (E)-2-decenal:

| | |
|---|---|
| Nonanal | 1.00 |
| Octanal | 0.32 ± 0.16 |
| Heptanal | 0.06 ± 0.03 |
| (E)-2-octenal | 0.04 ± 0.02 |
| (E)-2-decenal | 0.13 ± 0.065 |

3. The mosquito attractant composition of claim 1, having the following concentrations of heptanal, octanal, nonanal, (E)-2-octenal and (E)-2-decenal:

| | Concentration (μg/ml) |
|---|---|
| Nonanal | 47.6 ± 11.9 |
| Octanal | 15.429 ± 3.8575 |
| Heptanal | 2.857 ± 0.71425 |
| 2-octenal | 1.871 ± 0.46775 |
| 2-decenal | 6.136 ± 1.534 |

4. The mosquito attractant composition of claim 1, further comprising hexanal.

5. The mosquito attractant composition of claim 1, further comprising 1-octen-3-one.

6. The mosquito attractant composition of claim 1, not comprising 2-octanone.

7. The mosquito attractant composition of claim 1, further comprising an organic solvent.

8. An admixture comprising the mosquito attractant composition of claim 1, and a synthetic human or animal mosquito attractant blend.

9. The admixture of claim 8, wherein the mosquito attractant composition present in an amount of at least 0.5% (v/v) with the synthetic attractant blend.

10. The mosquito attractant composition of claim 1, wherein
the heptanal, octanal, nonanal, (E)-2-octenal and (E)-2-decenal are present in combination as more than 15% v/v of total volatiles in the composition, and/or
the heptanal, octanal, nonanal, (E)-2-octenal and (E)-2-decenal are present in combination as not more than 60% v/v of total volatiles in the composition.

11. (canceled)

12. A mosquito attractant composition comprising a natural human or animal odour source plus added heptanal.

13. The mosquito attractant composition of claim 12, wherein the added heptanal is present providing a total amount of heptanal which is about 10% greater (v/v) than the amount of heptanal in a natural human or animal odour obtained from Plasmodium-free human(s) or animals(s).

14. A composition comprising,
a natural or synthetic mosquito attractant, and
heptanal,
wherein when the composition is volatilised, heptanal is present in an amount of at least 0.7% v/v of all volatile odour compounds present.

15. The composition of claim 14, wherein heptanal is present in an amount of at least 0.9% v/v of all volatile odour compounds present.

16. The composition of claim 1, wherein heptanal is present in an amount of not more than 1.5% v/v of all volatile odour compounds present.

17. The mosquito attractant composition of claim 1, wherein the composition is in gaseous form.

18. A mosquito trapping composition comprising an adhesive substance and the mosquito attractant composition of claim 1.

19. The mosquito trapping composition of claim 18, further comprising an insecticide.

20. An apparatus for trapping or killing mosquitoes comprising:
(i) paper comprising the attractant composition of claim 1; and a CO2 emitting device comprising the paper;
(ii) a container or device comprising a porous medium or wax-like medium within the container or device, wherein the porous medium or wax-like medium comprises the attractant composition of claim 1;
(iii) a jar trap comprising the attractant composition of claim 1, wherein the jar trap dispenses carbon dioxide as an attractant;
(iv) a bug zapping device comprising the attractant composition of claim 1;
(v) an atomization device comprising the attractant composition of claim 1; or
(vi) an ionic dispersing device comprising the attractant composition of claim 1.

21. A method of detecting Plasmodium infection in a subject comprising:
(a) collecting a sample of odour emanated from the subject,
(b) detecting and measuring amounts of one or more indicative volatile compounds in the odour, the indicative volatile compound(s) selected from: heptanal, octanal, nonanal, (E)-2-octenal and (E)-2-decenal, 2-octanone, hexanal and 1-octen-3-one,
(c) comparing the measured amounts of the indicative volatile compounds with
i) the amounts of the same compounds in a reference sample of body odour from an uninfected subject or subjects; and/or
ii) predetermined reference amounts,
wherein an increase in the indicative volatile compound(s) indicates the subject has a Plasmodium infection.

22. The method of detecting Plasmodium infection of claim 21, wherein
the indicative volatile compounds are (E)-2-octenal and (E)-2-decenal and an increase in (E)-2-octenal and/or (E)-2-decenal compared to reference sample and/or reference amounts indicates that the subject is Plasmodium positive;
wherein the indicative volatile compounds are heptanal, octanal and nonanal, and the amount of increase in heptanal, octanal and nonanal compared to reference sample and/or reference amounts is proportional to the Plasmodium infection density in the subject; and/or
wherein the indicative volatile compound is selected from one or more of 2-octanone, hexanal and 1-octen-3-one and an increase in the indicative compound(s) compared to reference sample(s) thereof, and/or reference amount(s) is indicative of the presence of microscopic gametocytes in the subject.

23.-24. (canceled)
